(19) United States
(12) Patent Application Publication
(10) Pub. No.: US 2008/0312187 A1
(43) Pub. Date: Dec. 18, 2008

(54) PHOSPHOINOSITIDE MODULATION FOR THE TREATMENT OF ALZHEIMER'S DISEASE

(76) Inventors: Tae-Wan KIM, East Brunswick, NJ (US); Natalie Landman, Gilbert, AZ (US)

Correspondence Address: BAKER BOTTS L.L.P., 30 ROCKEFELLER PLAZA, 44TH FLOOR, NEW YORK, NY, 10112-4498, US

(21) Appl. No.: 11/934,534

(22) Filed: Nov. 2, 2007

Related U.S. Application Data
(63) Continuation of application No. PCT/US06/05745, filed on Feb. 17, 2006.
(60) Provisional application No. 60/736,735, filed on Nov. 14, 2005.
(60) Provisional application No. 60/735,311, filed on Nov. 12, 2005.
(60) Provisional application No. 60/677,133, filed on May 2, 2005.

(51) Int. Cl. A61K 31/661; C12N 9/99; C12Q 1/02; A61P 25/28

(52) U.S. Cl. 514/77; 435/184; 435/29; 514/114

(57) ABSTRACT

The present invention relates to methods of treating Alzheimer's Disease which utilize agents that increase neuronal phosphotidylinositol 4,5-biphosphate (PIP2), and to differentiated stem cell-based assay systems that may be used to identify agents that modulate phosphoinositide levels and thereby treat a variety of diseases. It is based, at least in part, on the discovery that edelfosine, an agent that increases PIP2 levels by inhibiting an enzyme that catalyzes PIP2 breakdown, decreases levels of neurotoxic A&bgr;42 peptide, particularly in cells expressing a mutant presenilin gene associated with Familial Alzheimer's Disease.

### PRIORITY CLAIM

This application claims priority to U.S. Provisional Application No. 60/736,735 filed Nov. 14, 2005; U.S. Provisional Application No. 60/735,311 filed Nov. 12, 2005, and U.S. Provisional Application No. 60/677,133 filed May 2, 2005, the contents of each of which is incorporated in its entirety herein.

### GRANT INFORMATION

The subject matter of this application was developed at least in part using National Institutes of Health Grant No. NS4346H, so that the United States Government holds certain rights herein.

### 1. INTRODUCTION

The present invention relates to the use of agents that increase phosphotidylinositol 4,5-biphosphate (PIP2) for the treatment of Alzheimer's Disease, MCI, and for improving memory, and to differentiated stem cell-based assay systems which may be used to identify agents that modulate phosphoinositide levels and thereby treat a variety of diseases.

### 2. BACKGROUND OF THE INVENTION

### 2.1 Alzheimer's Disease

Alzheimer's disease (AD) is the most common age-associated debilitating neurodegenerative disorder, affecting approximately 4 million Americans and about 20-30 million people worldwide. AD is characterized by a progressive decline in cognitive and functional abilities, and always results in death. The classical neuropathological features of AD include the presence of senile (β-amyloid-containing) plaques and neurofibrillary tangles (4) in the hippocampus, the amygdala, and the association cortices of the temporal, frontal and parietal lobes. More subtle changes include reactive astrocytic changes, as well as the loss of neurons and synapses in the entorhinal cortex and basal forebrain.

### 2.2 Presenilins and Familial Alzheimer's Disease

About five percent of AD cases are familial (FAD) and inherited by autosomal dominant mutations in APP and the presenilins (PS1 and PS2). Although some FAD cases occur due to mutations in the amyloid precursor protein (APP) itself, more than half of FAD cases and the most aggressive forms of FAD (with onset typically occurring at 40-50 years of age, but rarely developing in the second or third decade of life) are attributable to missense mutations in the PS1 gene, with more than 140 mutations identified thus far (1-3). The presenilins are multipass transmembrane proteins that localize predominantly to the endoplasmic reticulum (ER) and other intracellular compartments, with a small pool present at the plasma membrane (5,6). PS is initially synthesized as a 42-43 kDa holoprotein that undergoes proteolytic cleavage within the cytoplasmic loop connecting putative transmembrane segments 6 and 7. This endoproteolytic processing generates stable 27-28 kDa N-terminal and 16-17 kDa C-terminal fragments that combine to form an enzymatically active heterodimer (7-9). Presenilins have two conserved aspartyl residues, a feature of aspartyl proteases, within the PS transmembrane domains 6 and 7 (10) and aspartyl protease transition-state analog Inhibitors bind directly to PS1 and PS2 (11,12). Accumulating evidence suggests that the presenilins may serve as catalytic components of the γ-secretase complex, an unconventional aspartyl protease which mediates the cleavage of a growing number of type-1 membrane proteins, including APP.

### 2.3 Generation of Amyloidogenic Aβ42 Peptide

In the case of APP, γ-secretase mediates the C-terminal cleavage of the amyloid-β (Aβ) domain, thereby liberating Aβ/p3 from membrane-bound APP C-terminal fragments generated through ectodomain shedding by α-(ADAM10 and TACE) or β-secretase (BACE1). γ-secretase cleavage generates two major Aβ isoforms-Aβ40 and Aβ42. It has been well documented (14,15) that all mutations in PS1 and PS2 genes result in modulation of γ-secretase activity, leading to an elevation in the generation of the highly amyloidogenic and neurotoxic Aβ42 species, possibly at the expense of the more benign Aβ40 peptide.

### 2.4 Presenilins and Intracellular Calcium

All of the identified and examined PS mutations also disrupt intracellular Ca²⁺ homeostasis (24). The perturbations in calcium signaling are very consistent and may be used to predict FAD several years prior to symptom onset (16). Initial observations of the effect of PS mutations on calcium signaling were documented more than a decade ago by Ito et al. (17) who showed that inositol (1,4,5)-triphosphate (IP3)-mediated calcium release is potentiated in fibroblasts from patients with AD. Analysis of elementary calcium release events in Xenopus oocytes overexpressing the PS1 M146V mutant showed an increase in sensitivity to IP3, suggesting of abnormally elevated calcium ER levels (18). Abnormal, agonist stimulated, ER calcium release was also reported by Guo et al. (19) in PC12 cells overexpressing the PS1 L286V mutant. Enhanced bradykinin and thapsigargin-induced calcium responses were also observed in neurons derived from transgenic mice overexpressing mutant PSI (20). Intriguingly, PS is also reported to modulate capacitative calcium entry (CCE), which regulates the coupled process of IP3-mediated ER calcium release and ER store replenishing (21). Loss of PSI expression leads to potentiation of CCS, while FAD PSI mutations attenuate CCE and store-operated currents (21-23).

### 2.5 Phosphoinositide Signaling and Alzheimer's Disease

Phosphoinositides (“PIs”) serve as signaling molecules in a diverse=ray of cellular pathways (25-27) and aberrant regulation of PIs in certain cell types has been shown to promote various human disease states (47). PI signaling is mediated by the interaction with signaling proteins harboring the many specialized PI-binding domains, including Pleckstrin Homology (PH), epsin N-terminal homology (ENTH), Fabp/YOTB/Vac1p/EEA1 (FYVE), Phox homology (PX), and N-WASP polybasic motif domains (49-54). The interaction between these PI-binding domains and their target PIs results in the recruitment of the lipid-protein complex into the intracellular membrane.

PI signaling is tightly regulated by a number of kinases, phosphatases, and phospholipases. A schematic diagram showing the conversions among biologically relevant PIs is presented in FIG. 1. In the central nervous system, the levels of PIs in nerve terminals are regulated by specific synaptic kinases, such as phosphoinositol phosphate kinase type 1γ (PIPk1γ) and phosphatases, such as synaptojanin 1 (SYNJ1). PIP2 hydrolysis in the brain occurs in response to stimulation of a large number or receptors via two major signaling pathways: a) the activation of G-protein linked neurotransmitter receptors (e.g. glutamate and acetylcholine), mediated by PLCβs, and b) the activation of tyrosine kinase linked receptors for growth factors and neurotrophins (e.g. NGF, BDNF), mediated by PLCγ. The reaction produces two intracellular messengers, IP3 and diacylglycerol (DAG), which mediate intracellular calcium release and protein kinase C(PKC) activation, respectively. Moreover, localized membrane changes in PIP2 itself are likely an important signal as PIP2 is a known modulator of a variety of channels and transporters (30).

Reduced PI concentration in the temporal cortex of AD patients, as compared to controls, has been reported by Stokes and Hawthorne (63). Quantification studies aimed at comparing the levels of specific PLC isozymes in control and AD brains have reported aberrant accumulation of PLCδ1 and PLCγ1 in AD (31, 32). Studies of agonist-stimulated PIP2 hydrolysis in post-mortem human control and AD brain fractions (33-35) have shown reduced PIP2 hydrolysis in response to cholinergic and serotonergic PLC activation. Several neurotransmitters that act through the PI pathway have been shown to increase APP-α release (64,65), thereby blocking Aβ biogenesis.

Receptor-mediated metabolism of inositol phosholipids is known to produce a number of lipid second messengers involved in control of cell growth, apoptosis, ion-channel gating, etc. Thus, enzymes responsible for destruction of these second messengers and deactivation of the corresponding signaling pathways are essential for proper cellular function. Both, the PLC and PI-3 kinase signaling pathways contain such regulatory activities, responsible for removal of the 5-phosphate from the various inositol phospholipids to form downstream metabolites. Based on substrate specificity, inositol 5-phosphatases are characterized as type I or type II. Type I activity acts upon the soluble head-groups of Ins(1,4,5)P3 and Ins(1,3,4,5)P4, producing biologically inactive metabolites and thus defining the absolute and temporal limits of inositol polyphosphate accumulation. In contrast, type II 5-phosphatases have activity toward one or more phosphoinositides and (at least some of) the products of 5-phosphatase action, e.g., PtdIns(4)P and PtdIns(3,4)P2, have potential second messenger functions. A list of known inositol phosphatases is presented in Table 1, below.

### 3. SUMMARY OF THE INVENTION

The present invention relates to methods of, and compositions for, treating Alzheimer's Disease or Mild Cognitive Impairment and/or improving memory which utilize agents that increase neuronal phosphotidylinositol 4,5-biphosphate (PIP2), and to differentiated stem cell-based assay systems that may be used to identify agents that modulate phosphoinositide levels and thereby treat a variety of diseases. It is based, at least in part, on the discovery that edelfosine, an agent that increases PIP2 levels by inhibiting an enzyme that catalyzes PIP2 breakdown, decreases levels of neurotoxic Aβ42 peptide, particularly in cells expressing a mutant presenilin gene associated with Familial Alzheimer's Disease. Further, results of experiments performed on Aβ model systems have shown that (i) increasing PIP2 in hippocampal cells in vitro inhibited the synaptic dysfunction associated with increased Aβ42; and (ii) increasing PIP2 in Aβ model (PSAPP) mice improved the spatial memory of the mice, as demonstrated in a water-maze test.

The present invention further relates to methods of treating Alzheimer's Disease or Mild Cognitive Impairment and/or improving memory which utilize agents that are activators of PLCβ3 and/or PLCγ1. In specific non-limiting embodiments, such agents may be administered together with a ginsenoside, such as, but not limited to, Rk1 and/or (20S)Rg3. This aspect is based, at least in part, on the discovery that selective inhibition of PLCβ3 or PLCγ1 counteracts the Aβ42-lowering effect of (20S)Rg3.

In still further embodiments, the present invention relates to methods of treating Alzheimer's Disease and/or improving memory which target molecules modulated by PIP2, such as β-secretase. Such methods including treating Alzheimer's Disease by administering a compound which inhibits β-secretase.

### 4. BRIEF DESCRIPTION OF THE FIGURES

FIGS. 1A-C. Interconversion of phosphoinositides. (A) Phosphoinositol 4-phosphate (PI(4)P, or “PIP”) is converted to phosphotidylinositol 4,5-biphosphate (P1(4,5)P2, or “PIP2”) by phosphoinositol phosphate kinase type 1γ (PIPK1γ). PIP2 may be hydrolyzed by phospholipase C(PI-PLC, or “PLC”) to form inositol triphosphate (IP3) and diacylglycerol (DAG), or may be converted into phosphoinositol (3,4,5) triphosphate (PI(3,4,5)P3, or “PIP3”) by phosphoinositide kinase 3 (PI3-K). PIP3 may be converted to PIP2 by the phosphatase “Phosphatase and Tensin homolog deleted on chromosome Ten” (PTEN), and PIP2 may be converted to PIP by the phosphatase synaptojanin 1 (SYNJ1). (B) PIPK1γ and SYJN1 are major PtdIns(4,5)P2-metabolizing enzymes in the brain. TLC analysis of liposomes (Folch fraction) incubated in the presence of [γ32P]ATP and brain cytosols from indicated wild-type (WT) and knock-out (KO) animals. (C) Phosphoimaging quantitation of data presented in (B).

FIG. 2A-E. Changes in PIP2 levels correlate with Aβ42 biogenesis. PIP2 levels (A) and Aβ42 biogenesis (D) in HeLa cells overexpressing human APPsw treated with either PLC inhibitor edelfosine (EDEL) or its active analog miltefosine (MILT). PIP2 levels (B) and Aβ42 biogenesis (E) in HeLa cells overexpressing human APPsw treated with PLC activator m-3m3FBS (M3M). (C)

Full length APP and total Aβ biogenesis are not affected in treated cells.

FIG. 3A-F. PIP2 levels modulate Aβ biogenesis via two mechanisms. PIP2 levels modulate the release of soluble APP ectodomain into the medium. HeLa cells stably expressing APPsw were treated with either PLC inhibitors (EDEL, MILT) or PLC activator (M3M). Conditioned cell media were analyzed for secreted APP ectodomains generated by α-(sAPPα) (A) and β-secretase (sAPPβ) (B) cleavage. Aβ42 (D) and total Aβ biogenesis (C) in HEK293 cells transiently transfected with C99, C-terminal stub of APP that serves as a direct γ-secretase substrate, in the presence of PIP2 level modulator, EDEL. Aβ42 (F) and total Aβ biogenesis (E) in HEK293 cells transiently transfected with C99, C-terminal stub of APP that serves as a direct γ-secretase substrate, in the presence of PIP2 level modulator, M3M.

FIG. 4. Modulation of Aβ42 biogenesis by SYNJ1 and PIPK1γ. (A) Overexpression of SYNJ1 increases secreted Aβ42, Stable CHO-APP cells were transiently transfected with either vector (pcDNA3) or the HA tagged 5 phosphoinositol phosphatase domain of human synaptojanin1 (hSJ1-IPP). Top panel: Expression of hSJ1-IPP was assessed by Western blotting (HA). (B) Aβ42 levels (normalized to APP). (C) Total secreted Aβ. (D, E) PIPK1γ-90 and -87 isoforms decrease both the level of secreted Aβ42 and secreted total Aβ. Stable CHO-APP cells transiently transfected with human wild-type (PIPKIγ-90WT and PIPKIγ-87WT) and mutant (PIPKIγ-90 KD) PIPKIγ. Aβ42 values (D) and the corresponding total Aβ blot (E) are shown.

FIG. 5. SMT-3, a PIP2 modulator, blocks Aβ42 oligomer-induced synaptic dysfunction. The field excitatory post synaptic potential slope (FEPSP slope) in hippocampal slices that were untreated or treated with Aβ42 (Aβ) or Aβ42 and 20(S)Rg3 was monitored over time. Changes in fEPSP slope shows differences in long-term potentiation (LTP) expression in control hippocampal slices or hippocampal slices treated with either Aβ42 or combination of Aβ42 and 20(S)Rg3.

FIG. 6. PIP2 modulation improves spatial working memory impairment. PSAPP mice at 3 months of age were subjected to the radial-arm water-maze (n=3 per group). Wild-type mice at 3 months of age showed excellent performance during the acquisition of the task (A1-A4) and memory retention (R). In contrast, PSAPP mice exhibited working memory impairments. Treatment with edelfosine (EDEL; oral 1 mg/kg) improves memory retention of PSAPP mice.

FIG. 7A-B. Levels of various phospholipids in brains of wild-type (WT) and double knock-out (KO) PS½ mice, as measured by HPLC. (A) PI, DPG, PS and PA; (B) PIP and PIP2. DPG=diphosphoglycerate, PS=phosphatidyl serine, PA=phosphatidic acid.

FIG. 8A-B. PIP2 turnover is reduced in the presence of (A) PS1 and (B) PS2 FAD-associated mutations. Phosphoimage quantification of lipid kinase and TLC analysis of membranes prepared from HEK293 cells stably transduced with either wild-type (WT) or FAD mutant (ΔE9, L286V) PS1 (left panel) and wild-type (WT) or FAD mutant (N141I) PS2. P1(4,5)P2 is reduced by 26-40% in FAD expressing vs. WT-expressing cells.

FIG. 9. Inhibition of PLC, but not γ-secretase, reverses FAD-associated reduction in PIP2 turnover. HEK293 cells stably expressing either wild-type (WT) or FAD mutant (ΔE9, L286V) PS1 were pretreated with either DMSO, edelfosine (EDEL) or γ-secretase inhibitor (CpdE) for 6 hours prior to lipid kinase/TLC analysis.

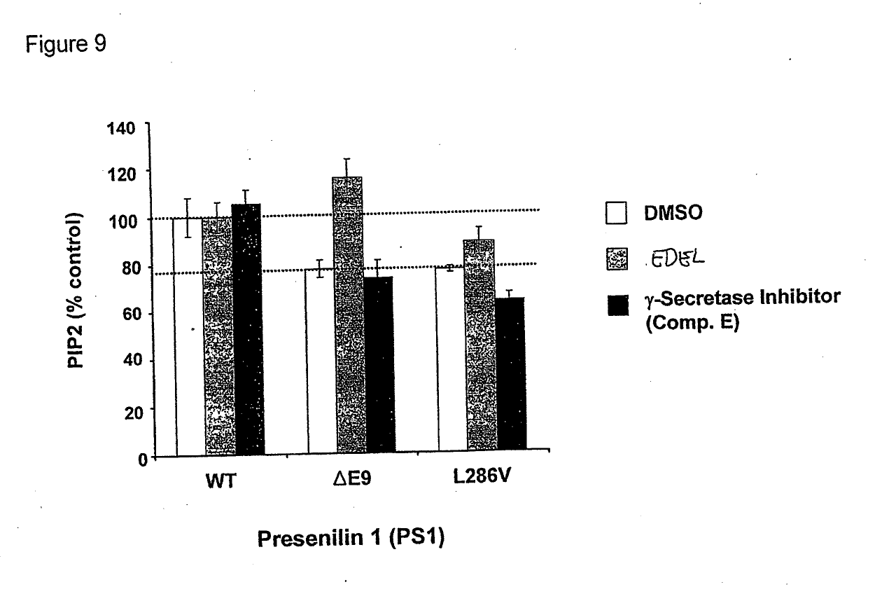

FIG. 10A-G. Directed differentiation of mouse embryonic stem (ES) cells into pyramidal neurons. (A) Phase-contrast image of ES-derived neurons at day 5 of differentiation. Limited variability in cell morphology suggests a very homogeneous cell population. (B) Immunocytochemical analysis of ES-derived neurons at day 8 of differentiation (left panel). Note that 90% of cells co-stain with DAPI and neuronal β-tubulin (TUJ-1). (C) Western blot analysis of cell lysates at different stages of differentiation. With onset of differentiation cells display a gradual increase in a variety of neuronal markers, e.g. TUJ-1 and synaptophysin, as well as pyramidal neuron-specific markers such as TrkB and CamKII. (D) ES-derived neurons form functional synapses, as indicated by FM 1-43 re-uptake assay, day 20. (E) cells of (D), loading with 90 mM KCl; (F) cells of (D), unloading with 90 mM KCl. (G) ES-derived neurons display depolarization-evoked activity characteristic of young hippocampal neurons, as measured by whole-cell voltage clamp recordings.

FIGS. 11A-E. Generation of mouse ES cells expressing human FAD-variants of PS1. (A) Mouse ES cells were stably transfected with either empty (vector) or FAD-PS1 (PS1ΔE9, PS1L286V, PS1M146V) containing plasmids by electroporation and subsequent antibiotic selection. (B-E) Clones were analyzed for human PS1 FAD expression using anti-human and anti-mouse PS1 antibodies.

FIG. 12. Expression of APP in ES-derived neurons transfected with lentivirus carrying the Swedish variant of human APP (hAPPsw). (A) Schematic of the hAPPsw-carrying lentiviral vector. (B) Full length APP (APP FL), as well as soluble fragment (sAPP) and total Aβ are easily detected in cell lysate and conditioned media, respectively. Control=untransfected.

FIG. 13. PS1—FAD expressing ES-derived neurons recapitulate the Aβ42 FAD-associated phenotype. Control (vector) or PS1ΔE9-expressing ES-derived neurons were transfected with lentivirus carrying hAPPsw. 48 hrs post infection conditioned media were analyzed for Aβ42 using sandwich ELISA. PS1ΔE9-expressing ES-derived neurons show a ˜10-fold increase in levels of secreted Aβ42, as compared to control neurons.

FIG. 14A-C. (A) Ginsenoside Rk1 selectively decreases Aβ42 relative to Aβ40. (B) Ginsenoside (20S)Rg3 also selectively decreases Aβ42 relative to Aβ40. (C) To a lesser extent, ginsenoside Rg5 selectively decreases Aβ42 relative to Aβ40.

FIG. 15A-B. (A) Rk1 and (20S)Rg3 decrease Aβ42 in cultured hippocampal primary neurons from Ad-model Tg2576 mice. (B) (20S)Rg3 decreases the ratio of Aβ42 to Aβ40 in the brains of Tg2576 mice.

FIG. 16A-B. CCE was induced in 293 cells stably expressing the mutant senilin, PS1ΔE9, in Ca²⁺-free medium containing Thapsigargin. (A) effect of increasing concentration of Rk1 on the F₃₄₀/F₃₈₀ ratio. (B) Effect of (20S)Rg3, (R)Rg3, Rk1, Rg5, Re and Rb2 on the F₃₄₀/F₃₈₀ ratio.

FIG. 17A-B. (A) γ-secretase inhibitor does not have a substantial effect on the F₃₄₀/F₃₈₀ ratio. (B) Aβ42-lowering NSAIDs tested do not have a substantial effect on the F₃₄₀/F₃₈₀ ratio.

FIG. 18A-B. Role of PLC γ1 in Aβ42-lowering activity of ginsenosides. (A) Hela-APPsw cells transfected with specific siRNA against PLCβ3, PLC γ1 and PLC γ2 were treated with either DMSO or 15 μM Rg3 for 6 hr. The down regulation of PLCβ3, PLCγ1 and PLCγ2 was confirmed by Western blot using isoform-specific antibodies. (B) Effects of RNAi-mediated downregulation of PLC isoforms in the presence of Rg3 treatment. Aβ42 levels were measured in the conditioned media by ELISA. Aβ values are shown as percentage of control siRNA and are the mean ±s.d. from three independent experiments (*P<0.001, **P<0.01 using ANOVA followed by Dunnett's test). (77,78)

### 5. DETAILED DESCRIPTION OF THE INVENTION

For clarity, and not by way of limitation, the detailed description of the invention is divided into the following subsections:

(i) methods of increasing PIP2 levels;

(ii) PIP2 modulated secretases as therapeutic targets;

(iii) assay systems to identify PIP2 modulators; and

(iv) methods of treating Alzheimer's disease, MCI, and/or improving memory.

### 5.1 Methods of Increasing PIP2 Levels

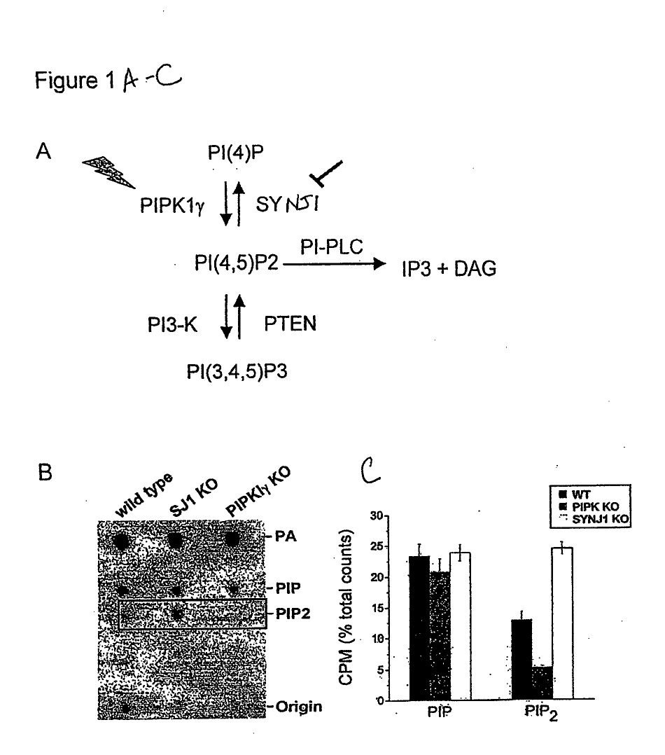

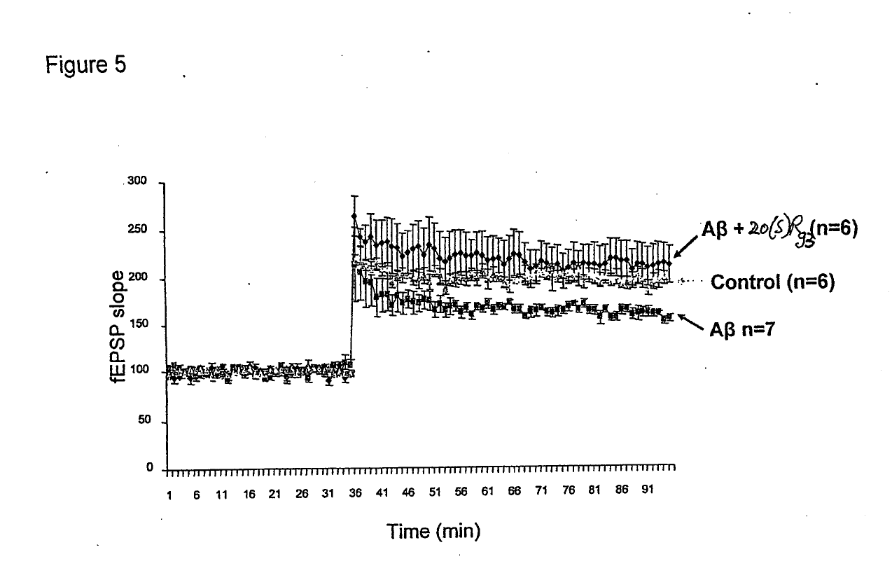

The present invention provides for methods of increasing PIP2 levels in a cell in need of such treatment, comprising administering, to the cell, an amount of an agent which modulates molecules involved in PI metabolism (e.g., see FIGS. 1A-C) and that preferably, but not by way of limitation, is effective in increasing PIP2 levels by at least about 5 percent, at least about 10 percent, and/or that is detectable by an assay system comprising a PI-sensor, as described below. Such an agent may, for example and not by way of limitation, increase the activity of PIPK1γ, inhibit the activity of PLC, inhibit the activity of SYNJ1, inhibit the activity of PI3-K, or increase the activity of PTEN. A “cell in need of such treatment” may be a cell involved in the pathogenesis of a condition associated with a defect in phosphoinositide signaling; e.g. a pancreatic β cell, a cancer cell (e.g., an acute myeloid leukemia cell), or, preferably, a neuron manifesting one or more features of AD, such as elevated Aβ42 production and/or level, senile plaques, neurofibrillary tangles, and/or synaptic dysfunction (e.g., a hippocampal neuron, see FIG. 5). Without limitation, desired effects of the present invention on a treated cell include, in addition to increased PIP2, a decrease in Aβ42, and/or an increase in long-term potentiation.

As a first example, the invention provides for the use of edelfosine, or a derivative thereof, at a concentration that inhibits PLC and that preferably, but not by way of limitation, increases intracellular PIP2 by at least about 5 percent or at least about 10 percent and/or by an amount that is detectable in an assay system comprising a PI sensor. In specific, non-limiting embodiments, edelfosine or its derivative may be administered to achieve a local concentration in the area of cells to be treated of between about 1 and 50 μM, and preferably between about 5 and 20 μM. In further specific, non-limiting embodiments, edelfosine or its derivative may be administered, to a human subject containing a cell to be treated, intravenously, subcutaneously, intrathecally, or by other methods known in the art, at a dose of about 15-20 mg/kg/day (61).

As a second example, the invention provides for the use of miltefosine, or a derivative thereof, at a concentration that inhibits PLC and that preferably, but not by way of limitation, increases intracellular PIP2 by at least about 10 percent and/or by an amount that is detectable in an assay system comprising a PI sensor. Miltefosine may be obtained from Zentaris, GmbH. In specific non-limiting embodiments, miltefosine or its derivative may be administered to achieve a local concentration in the area of cells to be treated of between about 3 and 25 μm. In further specific, non-limiting embodiments, miltefosine or its derivative may be administered, to a human subject containing a cell to be treated, orally, or intravenously, subcutaneously, intrathecally, or by other methods known in the art, at a dose of about 2.5 mg/kg/day, and/or a 10 mg or 50 mg tablet administered orally once or twice a day.

As a third example, the invention provides for the use of a phopholipid derivative as set forth in German patent DE 4222910, such as, but not limited to, perifosine, at a concentration that inhibits PLC and that preferably, but not by way of limitation, increases intracellular PIP2 by at least about 10 percent and/or by an amount that is detectable in an assay system comprising a PI sensor.

As a fourth example, the invention provides for the use of an erucyl, brassidyl or nervonyl-containing phosphocholine as set forth in European Patent No. 507337, such as, but not limited to, erucylphosphocholine, or a derivative thereof, at a concentration that preferably, but not by way of limitation, increases intracellular PIP2 by at least about 10 percent and/or by an amount that is detectable in an assay system comprising a PI sensor. In a specific, non-limiting example, erucylphosphocholine, or a related compound as set forth in European Patent Application No. 507337, may be administered, to a human subject containing a cell to be treated, orally, or intravenously, subcutaneously, intrathecally, or by other methods known in the art, at a daily dose of about 0.5-10 millimoles.

As a fifth example, the invention provides for the use of an alkylphosphocholine, including, but not limited to, the alkylphosphocholines disclosed in U.S. Pat. No. 4,837,023, e.g. hexadecylphosphocholine, or a derivative thereof, at a concentration that preferably, but not by way of limitation, increases intracellular PIP2 by at least about 10 percent and/or by an amount that is detectable in an assay system comprising a PI sensor. For example, said alkylphosphocholine may be administered, to a human subject containing a cell to be treated, orally, intravenously, subcutaneously, intrathecally, or by other methods known in the art, at a dose of about 5 to 2000 mg, and preferably between about 5 and 100 mg, per day.

As a sixth example, the invention provides for the use of ilmofosine, or a derivative thereof, at a concentration that inhibits PLC and that preferably, but not by way of limitation, increases intracellular PIP2 by at least about 10 percent and/or by an amount that is detectable in an assay system comprising a PI sensor. In further specific, non-limiting embodiments, ilmofosine or its derivative may be administered, to a human subject containing a cell to be treated, preferably intravenously, or by other methods known in the art, at a dose of about 12-650 mg/m² once per week (55), or preferably orally or subcutaneously (or by other methods known in the art) at a dose of about 10 mg/kg (56).

As a seventh example, the invention provides for the use of BN 52205 (57), or a derivative thereof, at a concentration that inhibits PLC and that preferably, but not by way of limitation, increases intracellular PIP2 by at least about 10 percent and/or by an amount that is detectable in an assay system comprising a PI sensor.

As an eighth example, the invention provides for the use of BN 5221.1 (57), or a derivative thereof, at a concentration that inhibits PLC and that preferably, but not by way of limitation, increases intracellular PIP2 by at least about 10 percent and/or by an amount that is detectable in an assay system comprising a PI sensor.

As a ninth example, the invention provides for the use of 2-fluoro-3-hexadecyloxy-2-methylprop-1-yl 2′-(trimethylammonio) ethyl phosphate (58) or a derivative thereof, at a concentration that inhibits PLC and that preferably, but not by way of limitation, increases intracellular PIP2 by at least about 10 percent and/or by an amount that is detectable in an assay system comprising a PI sensor.

As a tenth example, the invention provides for the use of the P13-K inhibitor, LY294002 (59,60), at a concentration that inhibits PI3K and that preferably, but not by way of limitation, increases intracellular PIP2 by at least about 10 percent and/or by an amount that is detectable in an assay system comprising a PI-sensor. In specific, non-limiting embodiments, LY294002 or its derivative may be administered to achieve a local concentration in the area of cells to be treated of between about 2 and 40 μM, and preferably between about 2 and 20 μM.

As an eleventh example, the invention provides for the use of a compound that inhibits a 5-phosphoinositide phosphatase, for example, but not limited to, a SYNJ1 inhibitor, including, but not limited to, Ro-31-8220 or Go-7874 Calbiochem/Novabiochem (Alexandria, Australia), or Inositol hexakisphosphate (InsP₆), at a concentration, for example but not by way of limitation, of 50 micromolar.

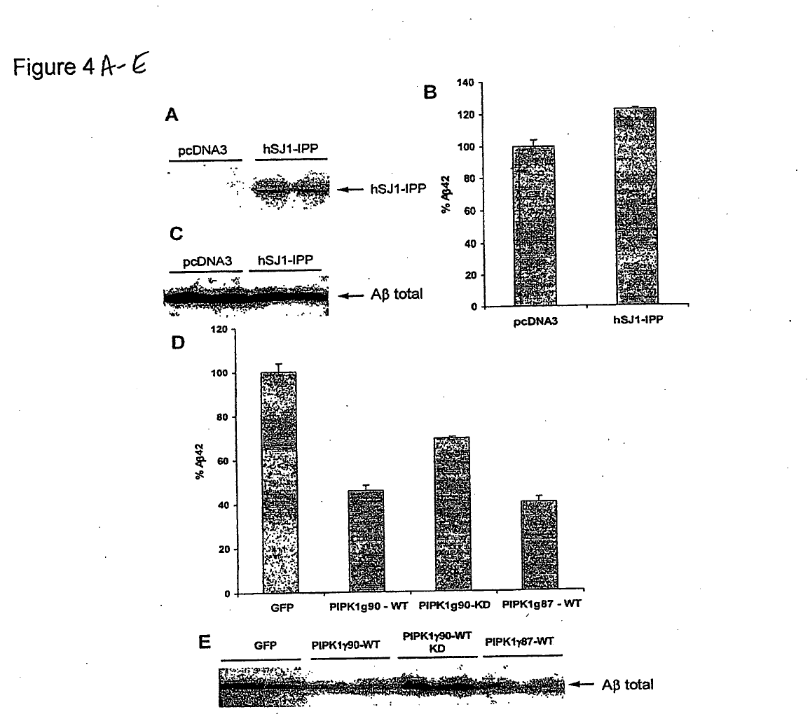

As a twelfth example, the present invention provides for the use of a compound that are agonists of PIP kinases (see FIGS. 4D and E).

### 5.2 PIP2-Modulated Secretases as Therapeutic Targets

In still further embodiments, the present invention relates to methods of treating Alzheimer's Disease, MCI and/or improving memory which target molecules modulated by PIP2, such as p-secretase. Such methods including treating Alzheimer's Disease or MCI and/or improving memory by administering a compound which inhibits β-secretase, including, but not limited to, compounds isolated from pomegranate as described in Kwak, H. M., et al, 2005. beta-Secretase (BACE1) inhibitors from pomegranate (Punica granatum) husk. Arch Pharm Res. 28(12):1328-32.

### 5.3 Assay Systems to Identify PIP2 Modulators

The present invention provides for assay systems and methods which may be used to identify compounds that either activate or inhibit modulators of phosphoinositides, including, but not limited to, PIP2.

In one set of embodiments, the present invention provides for an assay system for identifying an agent that modulates phosphoinositide levels in a differentiated class of cells, comprising a stem cell that expresses a detectable phosphoinositide sensor, wherein the stem cell is induced to differentiate in order to recapitulate one or more distinguishing features of the differentiated class of cells.

In another set of embodiments, the present invention provides for a method of identifying an agent that modulates the level of a phosphoinositide of interest, comprising:

(i) providing a stem cell that expresses a detectable phosphoinositide sensor (“PI sensor”) which binds to the phosphoinositide of interest, wherein the stem cell is induced to differentiate in order to recapitulate one or more distinguishing feature of the differentiated class of cells;

(ii) exposing the differentiated stem cell to a test agent; and

(iii) determining whether exposure to the test agent results in a detectable change in the phosphoinositide sensor;

wherein a change in the phosphoinositide sensor indicates that the test agent modulates the level of the phosphoinositide.

In other specific embodiments, as set forth below, the invention provides for an assay system for identifying an agent for treating Alzheimer's disease, comprising a stem cell induced to differentiate in order to recapitulate one or more distinguishing feature of a pyramidal neuron, optionally containing a PI sensor,

wherein the differentiated stem cell is engineered to further contain a gene associated with the development of Alzheimer's disease.

In the foregoing embodiments, the stem cell is preferably induced to differentiate into a cell type of interest. For example, for an assay system to identify agents that may be used to treat neurodegenerative diseases, the stem cell may be induced to differentiate to recapitulate a neuronal phenotype (“recapitulate” is used herein to mean that the differentiated cell shares one or more identifying feature, but not necessarily all phenotypic characteristics, of the cell of interest).

Where the assay system is used to identify agents for treating Alzheimer's disease, the stem cell is preferably induced to differentiate to recapitulate the phenotype of a pyramidal neuronl. Analogously, to identify an agent that may be used to treat Parldnson's disease, the stem cell may preferably be induced to differentiate to recapitulate the phenotype of a substantia nigral cell; to identify an agent that may be used to treat amyotrophic lateral sclerosis, the stem cell may preferably be induced to differentiate to recapitulate the phenotype of a motor neuron, etc.

The assay systems of the invention are not, however, limited to neuronal systems. Because phosphoinositides are associated with a diversity of diseases, the invention encompasses assay systems comprising stem cells induced to differentiate to recapitulate phenotypes of cells relevant to a disease of interest, such as, but not limited to, Islet cells to provide an assay system that may be used to identify agents that treat diabetes; cancer cells to provide an assay system that may be used to identify agents that treat cancer; cardiac cells to provide an assay system that may be used to identify agents that treat heart failure; hematopoietic stem cells to identify agents to treat transformed or hematopoietic cells with other abnormalities such as Myelodysplastic syndrome (MDS) or acute myeloid leukemia (AML); neuronal or astrocytic stem cells to identify the mechanism of formation and treatment of intracranial aneurysms; pulmonary stem cells to identify agents for treatment of asthma or COPD (chronic obstructive pulmonary disease) or muscle stem cells to identify agents for treatment of diseases such as X-linked myotubular myopathy (XLMTM) etc.

Sources of stem cells that may be used according to the invention include mouse (Evans and Kaufman, Nature. 1981, 292(5819):154-156; Martin, Proc Natl Acad Sci USA. 1981, 78(12):7634-8.), human (Thomson et al., Science. 1998, 282(5391):1145-1147; Shamblott et al., Proc. Natl. Acad. Sci. USA 1998 95:13726-13731), other mammalian non-human animals including but not limited to members of simian, bovine, feline, canine, equine, ovine, caprine or porcine species and chicken (Pain et al., 1996, Development 122, 2339-2348). Stem cells used according to the invention may be derived from various sources or growth stages including embryonic cells, fetal cells or adult stem cells. The invention includes but is not limited to a stem cell derived from cord blood; embryonic, fetal or adult neuronal stem cells; embryonic, fetal or adult hematopoietic stem cells; fetal or adult bone marrow stem cells; and stem cells derived from pancreatic ducts, intestine or hepatic cells. The invention also includes in a non-limiting embodiment fetal or adult mesenchymal stem cells derived from bone marrow or other tissues; endothelial progenitor cells; stem cells derived from adipose tissue; stem cells derived from hair follicles etc. The stem cell used in the invention may be a primary cell or an immortalized cell line. In specific embodiments the ES cells of the invention encompass but are not limited to mouse ES lines that stably overexpress the delta E9 and L286V mutant variants of human PS1. Another non-limiting example encompasses ES-derived pyramidal-like cells that express a variety of neuronal markers, including TUJ-1, CamKIIα, p75 and TrkB. A ES cell line expressing the Swedish variant of human APP (hAPPsw) may be utilized to recapitulate the Aβ42 generation phenotype.

The stem cell may be induced to differentiate using methods known in the art. The following is a non-limiting example of culturing stem cells for maintenance of the line or use in differentiation. A human stem cell (hSC) may be grown on gelatinized tissue culture dishes (0.1% gelatin coated) over a layer of mouse embryonic fibroblasts (CF1 strain), cultured in MEF medium, mitotically inactivated neuronal or astrocytic stem cells to identify the mechanism of formation and treatment of intracranial aneurysms; pulmonary stem cells to identify agents for treatment of asthma or COPD (chronic obstructive pulmonary disease) or muscle stem cells to identify agents for treatment of diseases such as X-linked myotubular myopathy (XLMTM) etc.

Sources of stem cells that may be used according to the invention include mouse (Evans and Kaufman, Nature. 1981, 292(5819):154-156; Martin, Proc Natl Acad Sci USA. 1981, 78(12):7634-8.), human (Thomson et al., Science. 1998, 282(5391): 1145-1147; Shamblott et al., Proc. Natl. Acad. Sci. USA 1998 95:13726-13731), other mammalian non-human animals including but not limited to members of simian, bovine, feline, canine, equine, ovine, caprine or porcine species and chicken (Pain et al., 1996, Development 122, 2339-2348). Stem cells used according to the invention may be derived from various sources or growth stages including embryonic cells, fetal cells or adult stem cells. The invention includes but is not limited to a stem cell derived from cord blood; embryonic, fetal or adult neuronal stem cells; embryonic, fetal or adult hematopoietic stem cells; fetal or adult bone marrow stem cells; and stem cells derived from pancreatic ducts, intestine or hepatic cells. The invention also includes in a non-limiting embodiment fetal or adult mesenchymal stem cells derived from bone marrow or other tissues; endothelial progenitor cells; stem cells derived from adipose tissue; stem cells derived from hair follicles etc. The stem cell used in the invention may be a primary cell or an immortalized cell line. In specific embodiments the ES cells of the invention encompass but are not limited to mouse ES lines that stably overexpress the delta E9 and L286V mutant variants of human PS1. Another non-limiting example encompasses ES-derived pyramidal-like cells that express a variety of neuronal markers, including TUJ-1, CamKIIα, p75 and TrkB. A ES cell line expressing the Swedish variant of human APP (hAPPsw) may be utilized to recapitulate the Aβ42 generation phenotype.

The stem cell may be induced to differentiate using methods known in the art. The following is a non-limiting example of culturing stem cells for maintenance of the line or use in differentiation. A human stem cell (hSC) may be grown on gelatinized tissue culture dishes (0.1% gelatin coated) over a layer of mouse embryonic fibroblasts (CF1 strain), cultured in MEF medium, mitotically inactivated by treatment with 10 μg/ml mitomycin C or inactivated by exposure to 8000 rads of γ-irradiation and plated at a density of 0.75×10⁵ cells/ml in 2.5 ml per well of a gelatin-coated 6-well dish. To passage the hSCs, cells may be washed once or twice with PBS and incubated with filter-sterilized 1 mg/ml collagenase IV in DMEM/F12 for 10 to 30 minutes. Plates may be agitated every 10 minutes until colonies begin to detach. When moderate tapping of the plate causes the colonies to dislodge, they may be collected and the wells washed with hSC medium to collect any remaining hSCs in the plate or well. Targeted differentiation of hSCs may be performed depending on the required type of lineage. The desired lineage may require choice of an appropriate hSC dependent on its known capacity to differentiate toward a specific lineage.

A non-limiting method to differentiate an undifferentiated neuronal progenitor stem cell is as follows. A neural progenitor cell may be converted to a dopaminergic neuron by incubation with retinoic acid (RA) (0.5 μM). The extent of differentiation may be followed by measuring the number of cultured cells showing positive immunoreactivity for the neuronal marker, microtubule-associated protein (MAP)-2ab, positive immunoreactivity to tyrosine hydroxylase (TH) and raised levels of dopamine (DA) and its metabolite, 3,4-dihydroxyphenylacetic acid (DOPAC) to indicate the presence of the dopaminergic neuronal phenotype. Brain-derived neurotrophic factor (BDNF) (50 ng/ml), glial-derived neurotrophic factor (GDNF) (10 ng/ml) and interleukin-1 beta (IL-1 beta) (10 ng/ml) may be used in the culture medium to promote neural progenitor cell differentiation towards the dopaminergic phenotype in the presence of dopamine (10 μM) and forskolin (Fsk) (10 μM). The trans-differentiation potential of the progenitor cells towards other neurotransmitter phenotypic lineages may also be achieved depending on the capacity of the stem cell. A suitable cocktail of agents, e.g. serotonin (Ser) (75 μM), acidic fibroblast growth factor (AFGF) (10 ng/ml), BDNF (50 ng/ml) and forskolin (10 μM, can direct certain human stem cell down a serotonergic cell lineage pathway determined by testing for tryptophan hydroxylase (TPH) positive immunoreactivity, and synthesis of 5-HT and its metabolites, secreted into the culture medium. Example Section 7, below, describes the targeted differentiation of murine ES cells to recapitulate the phenotype of pyramidal neurons.

Examples of cell types to be recapitulated by appropriate variations of the methods described above include, but are not limited to, neurons, glia, keratinocytes, dendritic cells, cardiomyocytes, hematopoietic cells, chondrocytes, pancreatic P-cells, adipocytes, osteoblasts, erythrocytes, vascular cells, skeletal muscle cells, hepatocytes, pneumocytes, and germ cells.

A PI sensor, according to the invention, is used to detect a change in PI level resulting from exposure of the differentiated cell, containing the sensor, to a test agent. Detection is preferably based on a change in cellular location of the sensor (see below), but may also be based on changes in other types of signal, for example, the intensity or frequency of a fluorescent signal, the generation of a reaction product, ability to bind to an epitope-specific antibody, etc. Thus, in non-limiting embodiments, detection and quantitation may be achieved by direct examination in live or fixed stem cells containing the PI sensor. Imaging techniques known to the art such as exposure to film, fluorescence microscopy, confocal microscopy or PhosphorImager methodology may be used to detect and measure the PI sensor. Alternatively, indirect means involving preparation of extract of the stem cell may be utilized to measure the amount of PI sensor. In alternative embodiments, after extraction from the stern cell, the PI sensor may be detected and quantitated using a specific detection reagent or system. The PI sensor may be measured directly after extraction if it is tagged or appropriately labeled. Alternatively the PI sensor may be indirectly measured through competition against a calibrated labeled competitor. The detection system whether for direct measure of the PI sensor or for the labeled competitor, in non-limiting embodiments, may be a fluorescent tag, a radioactive isotope, a specific epitope or coupled protein including but not limited to biotin, horseradish peroxidase, peptides such as HA-, Myc- or FLAG-tag etc. In a specific non-limiting embodiment the PI sensor may be detected and quantitated by equilibrium binding measurements utilizing protein-to-membrane fluorescence resonance energy transfer (FRET). This system detects domain docking to membrane-bound PIP lipids utilizing a physiological lipid mixture approximating the composition of the plasma membrane inner leaflet (Corbin et al. Biochemistry. 2004, 43(51):16161-16173).

Thus, the PI sensor of the invention typically is able to (i) bind phosphoinositide and (ii) generate a signal.

In one preferred, specific embodiment of the invention, the PI sensor is PH-GFP. See, for example, (62). The PH domain has a high affinity for PIP2 and localises to the plasma membrane, consistent with the known distribution of PIP2 in mammalian cells. The PH-GFP fusion protein provides a dynamic measure of PIP2 since activation of PLC and hydrolysis of PIP2 leads to a redistribution of PH-GFP from the plasma membrane to the cytosol. Conversely, an increase in PIP2, in the presence of PH-GFP PI sensor, leads to movement of and an accumulation of PH-GFP at the cell membrane, which can be visualized, for example, using fluorescence microscopy. Thus, an assay system of the invention comprising a PH-GFP PI sensor may indicate an increase in PIP2 by a localization of fluorescence (PH-GFP) at the cell membrane, so that the cell may appear to be brightly outlined.

In non-limiting embodiments of the invention, the PH-GFP molecule may comprise any suitable PH-domain sequence responsive to PI levels derived from a human or non-human animal source. Non-limiting examples of PH-domains include human DAPP1 (amino acids 167-257), human GRP1 (amino acids 267-399), mouse Btk PH domain (amino acids 6 to 217), Shc-PTB domain (amino acids 17-207) etc., fused either N-terminally or C-terminally to an appropriate GFP open reading frame. The present invention includes, in additional embodiments, PI sensor protein fusions encompassing a GFP-fluorescent tag fused with alternative PI-binding molecules including but not limited to appropriate FYVE (Fab1-YOYP-Vac1-EEAI) domains, ENTH (epsin amino-terminal homology) domains, PX (PLD2-Phox homology) domains, neural Wiskott Aldrich Syndrome protein (N-WASP) domains or other suitable PI binding domains known to the art. In another embodiment, the PI sensor based on any one of the PI-binding molecules set forth above may be fused to GFP related fluorescent proteins including but not limited to codon-optimized variants, enhanced variants and variants possessing different ranges of fluorescent emission spectra including for example a red-, blue- or yellow-fluorescent protein and variants thereof. The method of assay of the invention based on the PI sensor and used to detect a change in PI level resulting from exposure of the differentiated cell, containing the sensor, to a test agent is not dependent on specific identity or nature of interaction with PI. Thus in a specific embodiment the stem cell may be comprised of a PI sensor based on a PH, FYVE, ENTH, PX or N-WASP domain, fused to fluorescent protein or appropriately tagged as set forth above.

The detection and quantitation of the PI sensor is based on any one or more of the detection or assay systems set forth above. Additionally, the PI sensor encompasses all known mechanisms of interaction with PI including conformational change, intracellular localization change or other response dependent on the specific nature of the PI sensor interaction with PI.

The PI sensor of the invention may be incorporated into stem cells prior to targetted differentiation or afterward. A nucleic acid encoding the PI sensor may be prepared using standard techniques, and may optionally be comprised in a vector (see below) together with one or more element required or desirable for expression of the PI sensor in a cell including but not limited to promoter/enhancer elements, transcriptional and translational initiation and termination elements, other stabilization elements such as replication origins, intronic sequences, minigene sequences and/or a selectable marker. In particular embodiments, the promoter/enhancer elements used in the invention may comprise a tissue specific, cell type specific or developmental stage specific promoter, to further provide a differentiation-specific assay system. The selectable marker, when present, may include in non-limiting embodiments a neomycin, puromycin, blasticidin, hygromycin or zeocin resistance gene. The selectable marker may in particular embodiments be expressed utilizing the same transcriptional elements as the PI sensor (bicistronic conformation) or may be expressed via an independent set of expression elements.

Nucleic acid encoding the PI sensor of the present invention may be contained in a plasmid vector, a retroviral vector, an adenoviral vector, an adeno associated viral (AAV) vector or a lentiviral vector, comprising the aforementioned expression elements. In a specific embodiment a terminally differentiated neuron (day 7 in culture) may be transiently transduced to express a PI sensor of the invention using a lentiviral vector.

The present invention further comprises methods for delivering the PI sensor to a stem cell. Non-limiting examples of delivery methods include a physical means or a biological method. Thus a nucleic acid encoding the PI sensor, optionally contained in a vector, may be electroporated, microinjected, introduced by transfection including all variations known in the art of transfection or introduced by viral transduction utilizing viral vectors. The vectors of the invention may be integrating or episomal vectors. The vectors of the invention may additionally be either replicating or non-replicating plasmid or viral vectors. Alternatively, PI sensor protein may be introduced, for example, using liposome technology or other known means for promoting uptake of a protein into a cell.

Stem cells expressing PI sensor may also be transplanted into an animal in vivo to monitor changes in phosphoinositide levels in the animal as a result of administration of a test agent. In a particular embodiment, a stem cell expressing a transgenic PI sensor may be implanted into a pseudo-pregnant female mouse to generate a transgenic animal containing a PI sensor in all its cells. Such animals may then be utilized to isolate fresh populations of presumptive stem cell populations for further analyses. In a further embodiment, an appropriate promoter may be used to express the PI sensor in specific tissue, cell lineage or developmental stage and. Additionally, a heterologous stem cell expressing a PI sensor may be injected into an immunosuppressed animal system of a different species. The present invention encompasses but is not limited to the use of any of the above animal systems to detect a change in PI level resulting from exposure of the animal containing the sensor, to a test agent. A transgenic animal containing an integrated GFP-containing PI sensor in one or many of its cells or tissues or as a xenograft may be tested in vivo by appropriate means after administration of a test agent e.g. by monitoring GFP fluorescence in a live animal (Hansen et al In Vivo. 2002 16(3):167-174) or alternatively, tissue derived from such animals may be analyzed post-mortem. In a specific embodiment, PI sensor transgenic mice may be crossed with mouse models of Alzheimer's disease such as the 3×Tg-Aβ mice (Billings et al 2005 Neuron. 45(5):675-88) or other mouse models of human neurodegenerative diseases (Bloom et al, 2005 Arch Neurol. 62(2):185-187).

Using the assay systems of the invention, a universal phosphoinositide screening platform may be used to identify small molecule modulators of phosphoinositide effectors which are directly relevant to each target disease. Such technology provides a highly physiological cell system for drug discovery.

In further embodiments of the invention, differentiated stem cells as described above may be engineered to carry mutant forms of presenilin 1, presenilin 2, or β-amyloid precursor protein (APP), with or without a PI sensor, and used as model systems for AD and for use in assay systems to screen test agents for therapeutic efficacy against AD. Nucleic acid encoding genes for mutant forms of presenilin, APP, or other molecules associated with the etiology of AD may be introduced into such cells, for example by electroporation or transfection via a viral vector (e.g., a lentivirus or adeno-associated virus), either prior to, concurrent with, or following targetted differentiation. In related embodiments, stem cells, e.g. murine ES cells, harboring a germ-line M146V or other presenilin “knock-in” mutation may be prepared. Example section 7, below, describes the preparation of terminally differentiated neurons, prepared from murine ES cells, which are transfected with a lentivirus vector comprising the Swedish mutation of APP as well as the presenilin mutant, PS1-ΔE9; the present invention provides for such vectors, and model cells prepared therewith, using other, non-lentiviral vectors known in the art.

In still further embodiments, differentiated stem cells that recapitulate a neuronal, and particularly a pyramidal cell neuronal, phenotype may be used in a model system for AD whereby Aβ42 or Aβ soluble oligomers may be administerd to said cells, and then used to either (i) evaluate neuronal dysfunction, for example as measured by FM dye, calcium imaging or electrophysiology, and/or (ii) screen test agents as potential therapeutics for Aβ. Such Aβ42-exposed differentiated stem cells may optionally be engineered to further comprise a PI sensor, as set forth above.

### 5.3 Methods of Treating Alzheimer's Disease and/or Improving Memory

The present invention provides for a method of reducing Aβ342 generation in a neuronal cell (for example, in a human subject in need of such treatment) comprising administering, to the neuronal cell, an agent which (i) increases the amount of phosphoinositol 4,5 biphosphate (PIP2) and/or (ii) inhibits beta-secretase, in the neuronal cell. Examples of specific agents that may be used to increase PIP2 levels are set forth in Section 5.1 above, and assay systems for identifying further agents that may be so used are set forth in Section 5.3 above.

The present invention provides for methods of treating, preventing, or delaying the onset of AD or Mild Cognitive Impairment, “MCI” (and other neurodegenerative diseases associated with disorders in long term potentiation and/or with amyloid beta 42 accumulation), and/or for methods of improving memory, comprising administering, to a subject suffering from, or at risk of developing, said disorders and/or having impaired memory, an agent that increases neuronal levels of PIP2. A person at risk of developing AD includes persons with a family history of FAD, a person suffering from Mild Cognitive Impairment, or a person who has begun to exhibit other early signs of cognitive impairment associated with aging.

“Treating” as defined herein means conferring a clinical benefit and does not necessarily include improvement of cognitive abilities. For example, “treatment” includes a slowing or plateauing in the rate of cognitive deterioration.

“Improve (improving) memory” as defined herein includes subjective improvement of memory and/or objectively improved performance in a standard memory test (e.g., the Double Memory Test (Buscbke et al., 1997, Neurology 48:4989-4997), the Memory Impairment Screen (Buschke et al., 1999, Neurology 52:231), etc.).

Agents which may be used to treat AD, MCI and/or improve memory according to the invention include, but are not limited to, (i) edelfosine, or a derivative thereof, e.g., at a daily dose of between about 1-25 mg/kg/day and preferably between about 5-20 mg/kg/day, or in an amount to produce a local concentration in the brain of between 1 and 50 μM and preferably between 5 and 20 μM; (ii) miltefosine, or a derivative thereof, e.g., at a dose of about 2.5 mg/kg/day, and/or a 10 mg or 50 mg tablet administered orally once or twice a day; (iii) a phopholipid derivative as set forth in German patent DE 4222910, such as, but not limited to, perifosine; (iv) an erucyl, brassidyl or nervonyl-containing phosphocholine as set forth in European Patent No. 507337, such as, but not limited to, erucylphosphocholine, or a derivative thereof, e.g., at a daily dose of about 0.5-10 millimoles; (v) an alkylphosphocholine, including, but not limited to, the alkylphosphocholines disclosed in U.S. Pat. No. 4,837,023, e.g. hexadecylphosphocholine, e.g., at a dose of about 5 to 2000 mg, and preferably between about 5 and 100 mg, per day; (vi) ilnofosine, or a derivative thereof, e.g., at a dose of 12-650 mg/m²/week or 10/mg/kg per day; (vii) BN 52205 or a derivative thereof; (viii) BN 5221.1 or a derivative thereof, (ix) 2-fluoro-3-hexadecyloxy-2-methylprop-1-yl 2′-(trimethylammonio) ethyl phosphate or a derivative thereof, and (x) LY294002 or a derivative thereof, e.g., at a dose that provides a local concentration of 2-40 μM. The foregoing dosages are provided as examples and do not limit the invention as regards effective doses of the recited compounds.

In other particular, non-limiting embodiments, the present invention provides for a method of treating or preventing AD or MCI and/or improving memory comprising administering, to a subject in need of such treatment, a composition comprising an effective amount of an activator of PLCγ1. In non-limiting embodiments, the activator of PLCγ1 may be administered together (sequentially or contemporaneously) with an effective amount of an agent selected from the group consisting of Rk1, (20S)Rg3 and Rg5 or a combination thereof, preferably (20S)Rg3. In this latter context, “an effective amount” of each component is considered in the context of the various components acting together to produce an objective or subjective therapeutic benefit. Non-limiting examples of agents that activate PLCγ1 include agents that increase its level of expression or increase the activity of a single molecule.

In particular, non-limiting embodiments, the present invention provides for a method of treating or preventing AD or MCI and/or improving memory comprising administering, to a subject in need of such treatment, a composition comprising an effective amount of an activator of PLCβ3. In non-limiting embodiments, the activator of PLCβ3 may be administered together (sequentially or contemporaneously) with an effective amount of an agent selected from the group consisting of Rk1, (20S)Rg3 and Rg5, or a combination thereof, preferably (20S)Rg3. In this latter context, “an effective amount” of each component is considered in the context of the various components acting together to produce an objective or subjective therapeutic benefit. Non-limiting examples of agents that activate PLCβ3 include agents that increase its level of expression or increase the activity of a single molecule.

The present invention further provides for methods of treating, preventing, or delaying the onset of AD (or Mild Cognitive Impairment, “MCI”) and/or improving memory comprising administering, to a subject suffering from memory impairment and/or AD or MCI, or at risk of developing AD or MCI, an agent that modulates the levels of β-secretase activity. In a non-limiting embodiment, agents which modulate the activity of β-secretase can be identified by their ability to increase or decrease the levels of soluble APP ectodomain generated by β-secretase (sAPPB).

In other particular, non-limiting embodiments, the present invention provides for a method of treating or preventing AD or MCI comprising administering, to a subject in need of such treatment, a composition comprising an effective amount of an agent which prevents, treats, or delays the onset of Aβ42 oligomer-induced synaptic dysfunction and/or which promotes long term potentiation. Aβ oligomers can inhibit long-term potentiation and exhibit neurotoxicity and lead to synaptic dysfunction, which is a pathology associated with AD. Agents which prevent, treat, or delay the onset of Aβ42 oligomer-induced synaptic dysfunction can effect an increase in long-term potentiation (LTP) in neuronal cells, and accordingly can be useful in the prevention and treatment synaptic dysfunction associated with Aβ or MCI (FIG. 5). Long-term potentiation refers to the increase in action potentials of hippocampal neurons which are exposed to repeated stimuli from the same source, and play an important role in the formation of long-term memory. AD is often associated with impairment in LTP in hippocampal neurons, and in some cases Aβ42 oligomers may induce synaptic dysfunction by impairing LTP, resulting in impaired ability to form long term memory. Agents which prevent, treat, or delay the onset of Aβ42 oligomer-induced synaptic dysfunction can be identified by their ability to increase long-term potentiation (LTP), measured, for example, by changes in fEPSP slope. In a non-limiting embodiment, a potential agent will maintain or increase the LTP in a neuronal cell in the presence of Aβ42, relative to a control neuronal cell which is not treated with the agent or with Aβ342. Non-limiting examples of agents that prevent, treat, or delay the onset of Aβ42 oligomer-induced synaptic dysfunction include 20(S)Rg3.

In other particular, non-limiting embodiments, the present invention provides for a method of treating or preventing AD or MCI and/or improving memory comprising administering, to a subject in need of such treatment, a composition comprising an effective amount of an inhibitor of 5-phosphoinositide phosphatase. It has been found that inhibition of a 5-phosphoinositide phosphatases can result in a decrease in Aβ42 formation, and accordingly can be useful for the prevention or treatment of AD or MCI. Non-limiting examples of 5-phosphoinositide phosphatases include, but are not limited to: SynJ1, SynJ2, INPP5P, OCRL, SHIP1, SHIP2, SKIP, PIPP, Pharbin/INPP5E, PTEN, MINPP1, INPP1, SAC1, Sac2, and Sac3.

The present invention further provides for a method of identifying an agent that may have therapeutic benefit in the treatment of AD and/or MCI and/or, comprising identifying an agent that selectively activates (as defined above) isoform PLCβ3 and/or PLCγ1 of phospholipase C, which may be administered in conjunction with a ginsenoside, such as, but not limited to, 20(S)Rg3, Rk1, or Rg5.

The present invention provides for pharmaceutical compositions comprising effective amounts of the foregoing compounds, separately or in combination, in a suitable pharmaceutical carrier. The foregoing agents/compounds may be administered orally, intravenously, subcutaneously, intramuscularly, intranasally, intrathecally, or by any other method known in the art, as would be appropriate for the chemical properties of the compound.

### 6. WORKING EXAMPLE

### Effects of Modulating PIP2 Levels on Amyloid-Beta42 Production

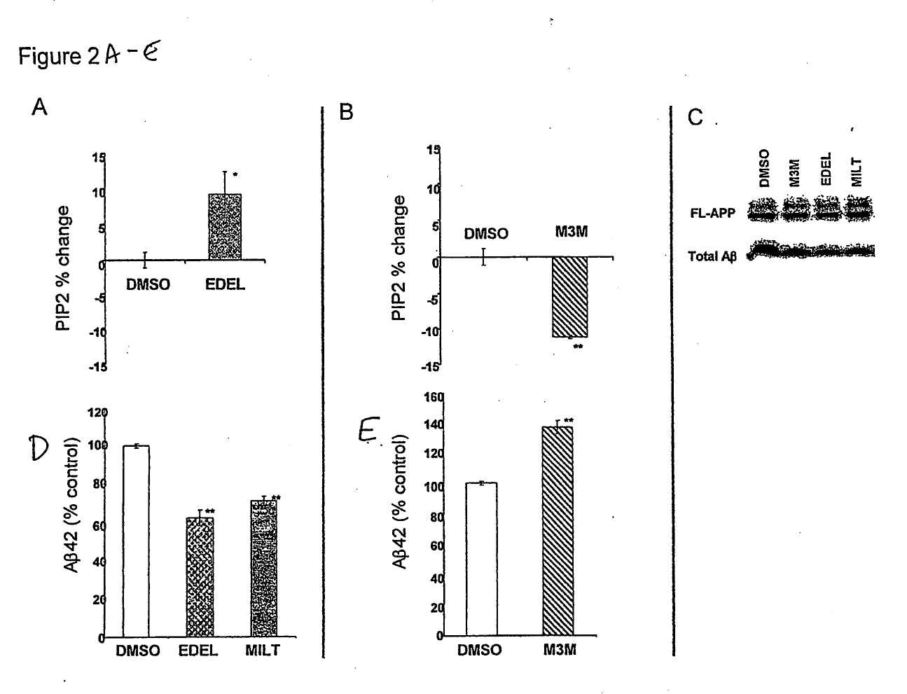

Modulation of PIP2 levels correlates with Aβ42 biogenesis. HeLa cells stably overexpressing the Swedish variant of human APP were treated with either control (DMSO), PLC inhibitor edelfosine (EDEL) or its active analog miltefosine (MILT). Steady-state PIP2 levels were determined by HPLC. As shown in FIG. 2A, treatment with edelfosine resulted in ˜10% increase in the steady-state levels of PIP2, with a corresponding 37.3% decrease in the levels of Aβ42 (FIG. 2D). Treatment with the PLC activator m-3m3FBS (M3M) resulted in ˜11% decrease in the steady state levels of PIP2 (FIG. 2B), with a corresponding 37.2% increase in Aβ42 (FIG. 2E). No significant effects of either treatment were observed on the steady-state levels of full-length APP (FL-APP), as determined by Western blot analysis (FIG. 2C).

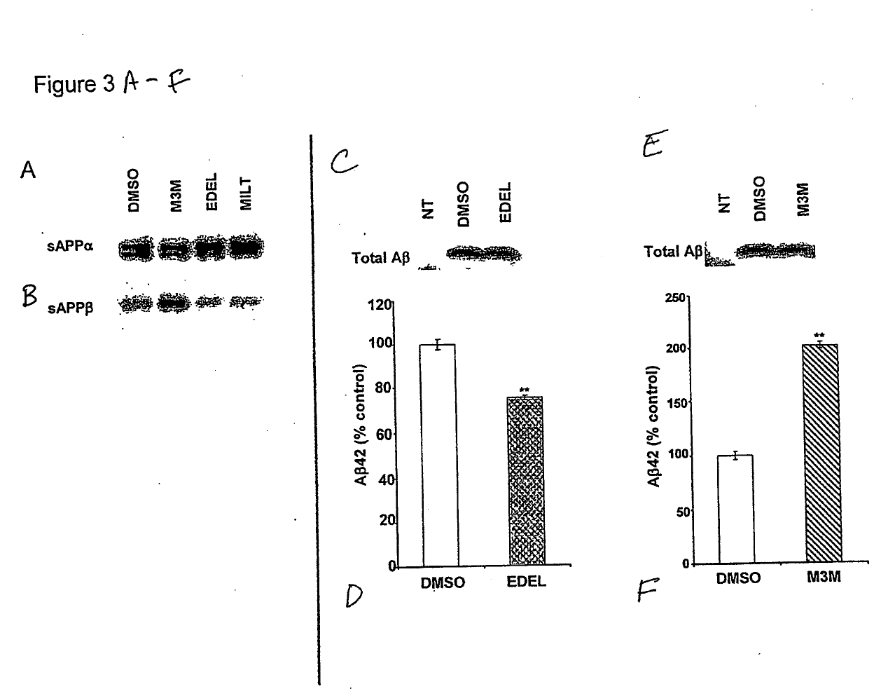

PIP2 levels modulate Aβ biogenesis via two distinct mechanisms. The metabolites of PIP2, including IP3 and DAG, have been implicated in APP processing pathways (69, 70, 71). Under normal conditions, PIP2 hydrolysis (to generate IP3 and DAG) favors the generation of α-secretase-generated secreted APP ectodomain (sAPPα). As predicted from previous studies, treatment with edelfosine (EDEL) or miltefosine (MILT) resulted in increases in sAPPα generation (FIG. 3A) with corresponding reduction in sAPPβ secretion (FIG. 3B). Interestingly, treatment with m-3m3FBS (M3M)led to a dramatic increase in β-secretase-mediated liberation of soluble APP (sAPPβ) with correlated decreases in sAPPα. To more clearly define the role of PIP2 in modulating γ-secretase activity (e.g. Aβ42), we next expressed an APP-C99 construct, an ectopic γ-secretase substrate which resembles β-secretase-generated, membrane-associated APP stub in heterologous cells. In C99-transfected cells, Aβ42-reducing activity of edelfosine and Aβ42-promoting activity of m-3m3FBS were still observed (FIG. 3C-F), indicating that P1(4,5)P2-mediated modulation of Aβ42 occurs at the level of preseilin/γ-secretase modulation.

Effect of other modulators of PIP2 on Aβ42 production. Synaptojanin 1 (SYNJ1) and PIP kinase type 1-γ (PIPK1γ) represent the major P1(4,5)P2-metabolizing enzymes in the brain (FIGS. 1A and B). SYNJ1 expression was previously shown to reduce the levels of cellular PIP2 (72). In contrast, overexpression of PIPK1γ in the cells is known to cause the elevation of cellular PIP2 levels (73). We, therefore, determined the effects of SYNJ1 or PIPK1γ on Aβ42 biogenesis (FIG. 4A-E). Expression of SYNJ1 constructs (containing a membrane targeting signal) caused increased generation of Aβ42 (FIG. 4B). Meanwhile, wild-type PIPK1γ expression (both γ90 and γ87 forms) lead to a substantial reduction in Aβ42 generation (FIG. 4D). In contrast, the kinase-dead mutant version of PIPK1γ did not confer any Aβ42-reducing activity, indicating that PIPK1γ-mediated Aβ42 reduction requires intact lipid kinase activity. Thus, modulation of PIP2 and Aβ42 by PIPK1γ or SYNJ1 to favor the augmentation of PIP2 (and corresponding Aβ42 decrease) may provide a novel therapeutic opportunity for treating Aβ-affected brain. These results also indicate that the PIP2 level is a critical determinant of Aβ42 biogenesis, since in our hands, any enzymatic reaction that favors P1(4,5)P2 synthesis leads to decreased Aβ42. Similarly, any enzymatic reaction that favors P1(4,5)P2 breakdown leads to increased Aβ42.

PIP2 modulation rescues Aβ oligomer-induced synaptic dysfunction. Soluble Aβ oligomers have been recently implicated in cognitive dysfunction prior to the formation of senile plaques, as soluble Aβ concentration in the brain shows a stronger correlation with cognitive dysfunction (74) and synapse loss (75). Moreover, Aβ oligomers can inhibit long-term potentiation and exhibit neurotoxicity (76). FIG. 5 shows that treatment with (20S)Rg3 (SMT-3), which has been shown to modulate PIP2 levels and reduce Aβ42 biogenesis, blocks Abeta oligomer-induced inhibition of long-term potentitation. FIG. 5 shows that treatment of hippocampal slices with Aβ42 reduces LTP expression relative to untreated hippocampal slices, as shown by the decrease in fEPSP slope. Addition of 20(S)Rg3 in the presence of Aβ42 increases LTP expression relative to untreated hippocampal slices.

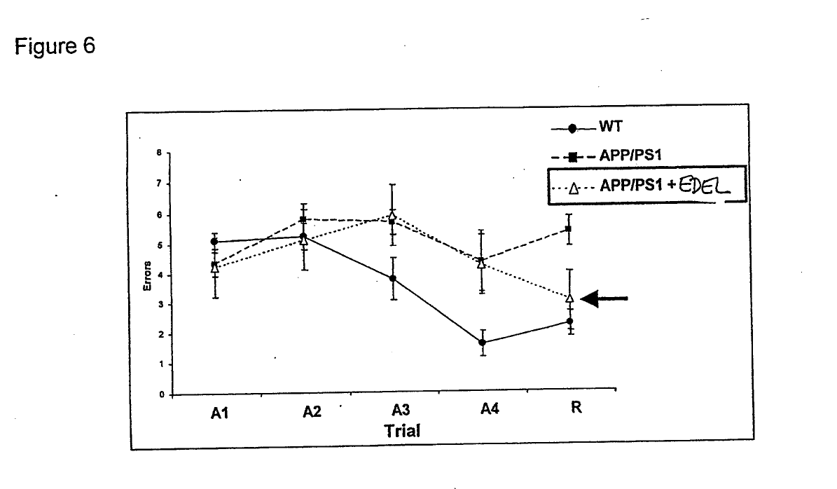

PIP2 modulation improves spatial working memory impairment. Double transgenic mice overexpressing the Swedish variant of amyloid precursor protein and PS1 FAD mutation (PSAPP) and wild-type littermates at 3 months of age were subjected to the radial-arm water-maze test (n=3 per group). As shown in FIG. 6, wild-type mice at 3 months of age showed excellent performance during the acquisition of the task (A1-A4) and memory retention (R). In contrast, PSAPP mice exhibited working memory impairments. Treatment with edelfosine (SMT-1) improved memory retention of PSAPP mice (arrow).

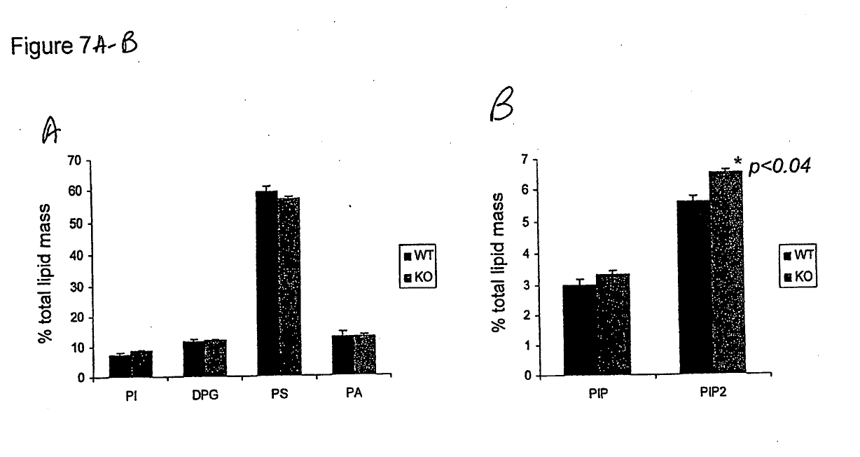

Presenilin deficiency modulates levels of PIP2 in the brain. In addition, levels of various phopholipids were measured by HPLC in the brain of wild-type and double knockout PS1/PS2 mice, to determine the effects of presenilin deficiency on PIP2 levels in vivo. As shown in FIG. 7A-B, levels of PIP2 were increased by 20 percent (statistically significant) in knock-out brain tissue as compared to control (p<0.04). Thus, presenilin deficiency (primarily in neurons) leads to significant elevation of PIP2 in the brain.

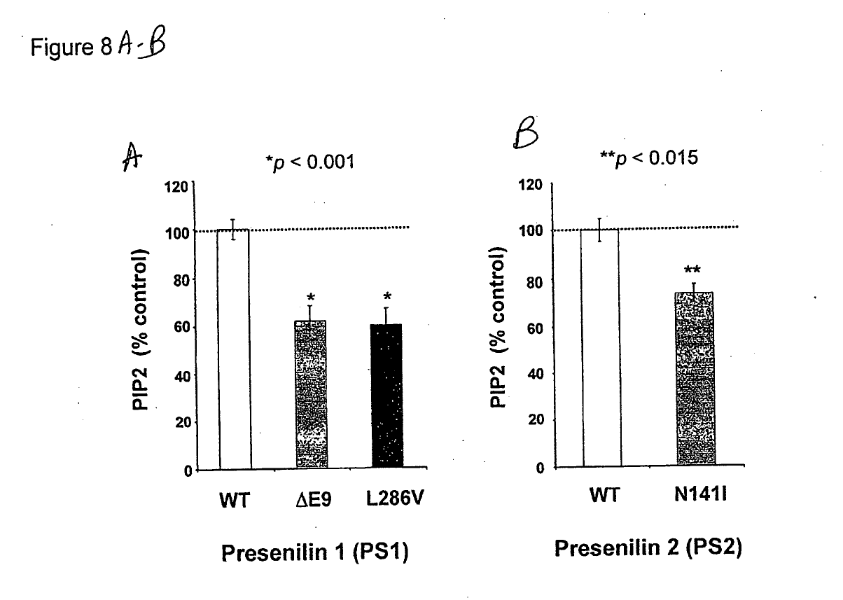

Expression of FAD mutant PS1 and PS2 isoforms results in abberant PIP2 metabolism. The role of the presenilins in PIP2 metabolism was further confirmed by observation that PIP2 turnover, as measured by radio-labeled lipid kinase/TLC assay, is reduced in PS1 (ΔE9, L286V) and PS2 (N141I) FAD expressing cells as compared to control (WT PS1/PS2) expressing cells. Phosphoimage quantification of 3 independent experiments shows that the conversion of radiolabeled phosphoinositides into P1(4,5)P2 is selectively reduced in FAD cells (26-40% reduction) as compared with wild-type cells (FIG. 8A-B). These results indicate that presenilin FAD mutations lead to either diminished synthesis or enhanced breakdown of PI(4,5)P2. Inhibition of PLC, but not γ-secretase, reversed FAD-associated reduction in PIP2 turnover.

Discussion, Phosphoinositides serve as signaling molecules in a diverse array of cellular pathways, and aberrant regulation of phosphoinositides in certain cell types can lead to various human disease states (47). A number of druggable molecular targets in the PI pathway have been suggested, including lipid phosphatase inhibitors (for diabetes), lipid kinase inhibitors, lysophospholipase D inhibitors, lipid recognition domain antagonists (cancer) and LPA receptor antagonists (for metastasis). The results demonstrate that regulation of phosphoinositides is critically associated with the pathogenesis of Alzheimer's disease.

Edelfosine (ET-18-OCH₃) is a synthetic analog of lysophophatidylcholine (etherphospholipid) which is known to modulate intracellular signaling and has been studied and/or used to treat cancer and infectious diseases (66). In the experiments described herein, it was found that treatment of various cell lines with edelfosine led to the reduction of Aβ42 observed in FAD cells. Miltefosine was shown to have similar effects. Thus, edelfosine, other etherphospholipid analogs and their chemical derivatives can be used to treat Alzheimer's disease and other neurodegenerative diseases.

### 7. WORKING EXAMPLE

### Preparation of Cells for Use in an Assay System for Identifying PIP2 Modulators

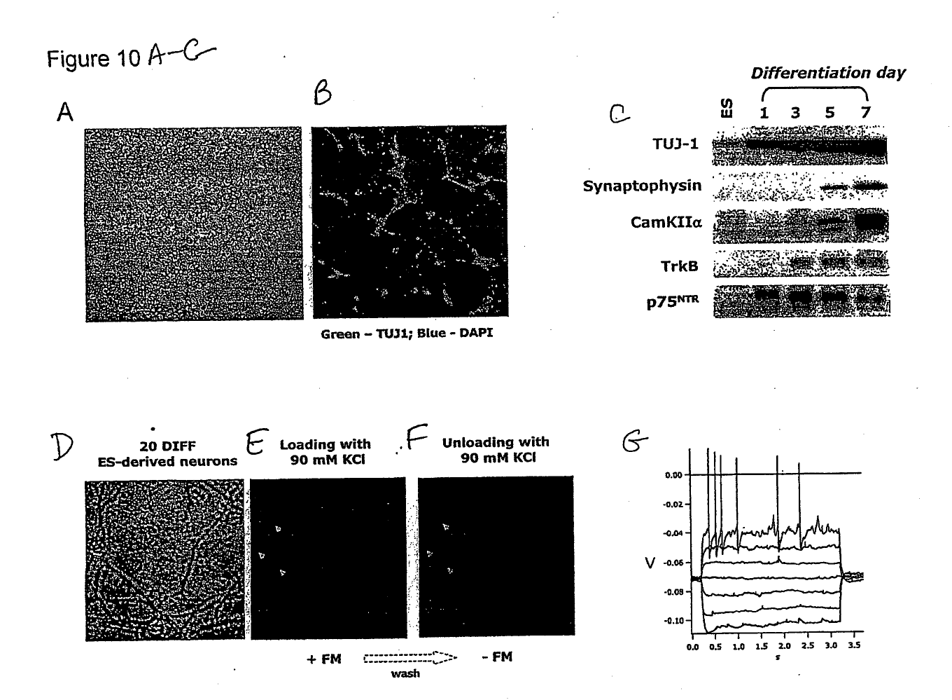

Targeted differentiation of wild-type mouse embryonic stem cells was performed by the method of Bibel (42), with the following modifications: 15% FBS, rather than FCS, together with added nucleosides (using premixed 100× solution purchased from Specialty Media (catalogue number ES-008-D)), were used in ES medium. Further, Neurobasal medium with penicillin/streptomycin, L-glutamine, and B27 supplement (Invitrogen) was used as the final differentiation medium, while Bibel et al. use a modified version of “B18 medium” described in Brewer et al. (48). This method produced neurons with pyramidal cell properties (FIG. 10A-D).

FIG. 10A shows ES-derived neurons at day 5 of differentiation. Limited variability in cell morphology suggests that the differentiation protocol used produced a very homogeneous cell population. Immunofluorescent studies (FIG. 10B) and analyses of cell lysates (FIG. 10C) show that these cells display a variety of neuronal markers (e.g., TUJ-1 and synaptophysin), as well as pyramidal neuron-specific markers such as TrkB and CamKII. ES-derived neurons form functional synapses, as indicated by FM 1-43 reuptake assay (FIGS. 10D-F) and display electrophysiological properties characteristic of young hippocampal neurons (FIG. 10G).

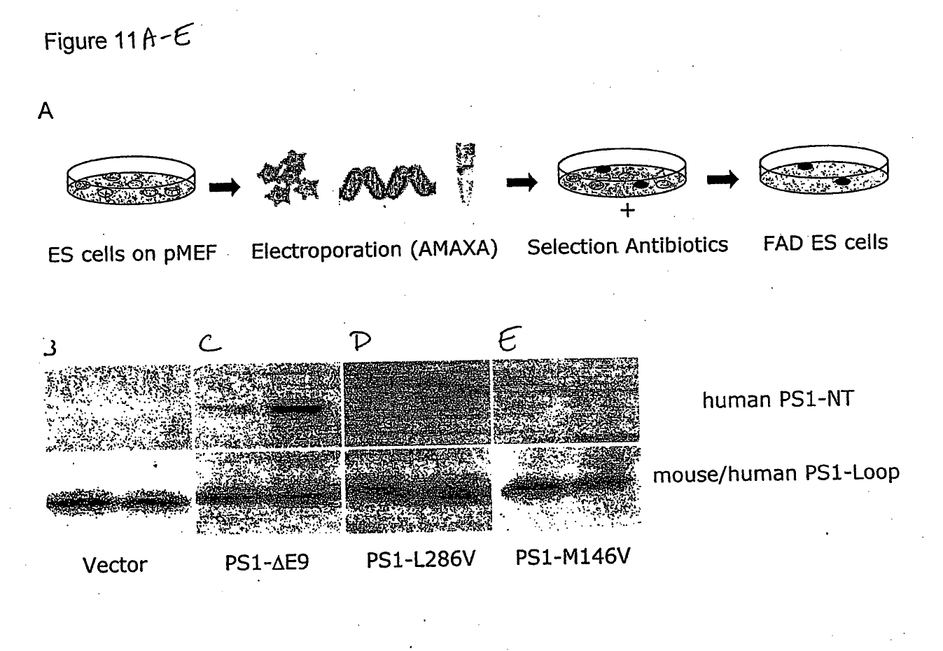

Mutant human presenilin genes associated with FAD were introduced into ES cells by electroporation (by the AMAXA Nucleofector system (Amaxa Gmbh, Cologne, Germany) (FIG. 11A). Expression of various presenilin mutants was achieved (FIG. 11B-E), with greater expression of PS1-ΔE9. Expression of PS1-ΔE9 presenilins did not appear to have an effect on differentiated ES cell morphology or on the expression of neuronal/pyramidal cell specific proteins.

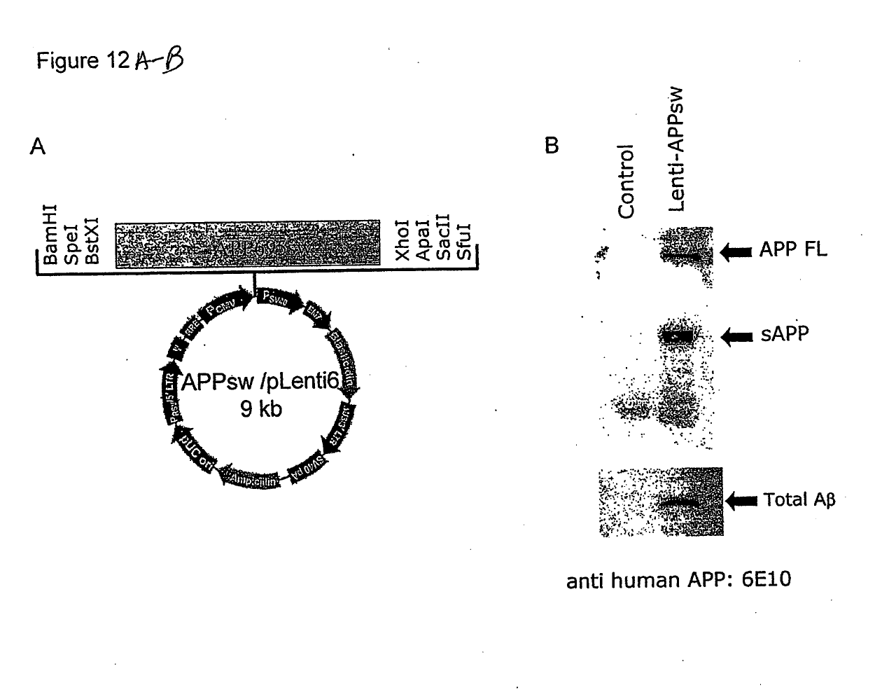

In addition, in order to recapitulate the Aβ42 generation phenotype, the Swedish variant of human APP (hAPPsw) was transiently expressed in terminally differentiated ES-derived pyramidal-like cells expressing TUJ-1, CamKIIα, p75 and TrkB (day 7 in culture) using a lentiviral vector (FIG. 12A). Using a human-specific anti-APP antibody (6E10, Sygnet), expression and proteolytic processing of hAPPsw in these cells was confirmed by Western blotting (see FIG. 12B). Untransfected ES-derived neurons were utilized as a control.

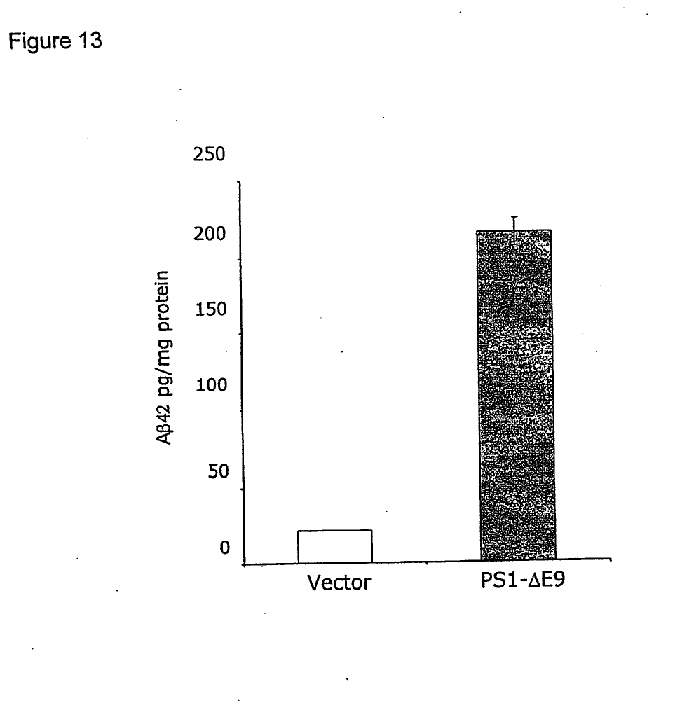

Aβ42 levels in differentiated ES-derived neurons transfected with Lenti-APPsw vector in the presence or absence of PS1-ΔE9 was compared FIG. 13). Aβ42 levels were found to be increased in differentiated ES-derived neurons co-expressing PS1-ΔE9. This data indicates that the differentiated ES cells coexpressing mutant presenilin and human APP recapitulate FAD-associated phenotypes, in particular Aβ42 generation. These cells were also found to exhibit reduced viability.

### 8. WORKING EXAMPLE

### Natural Compounds Derived From Heat-Processed Ginseng Reverse Molecular Phenotype Associated with FAD-Linked Presenilin Mutations

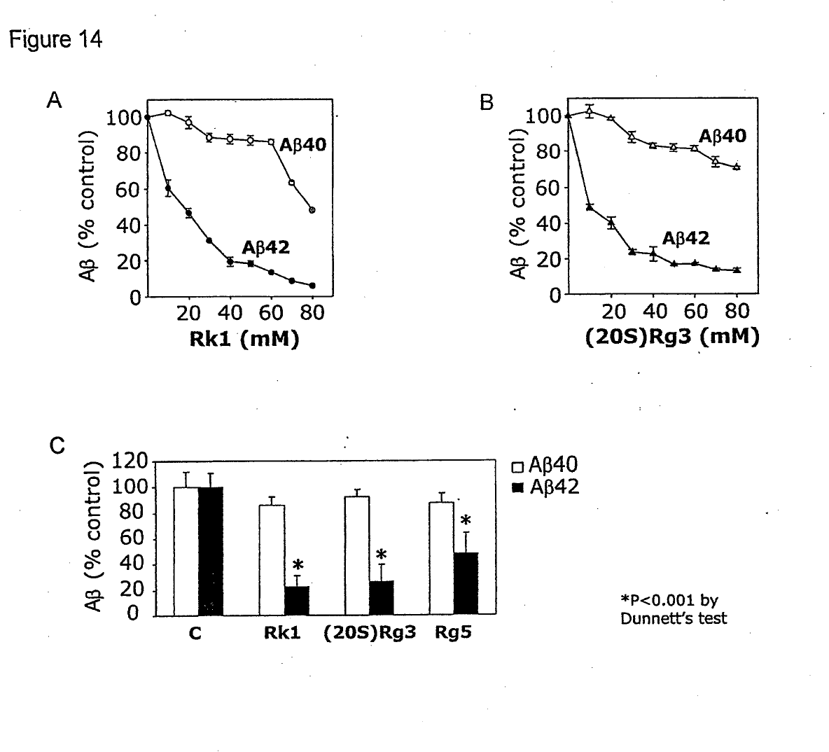

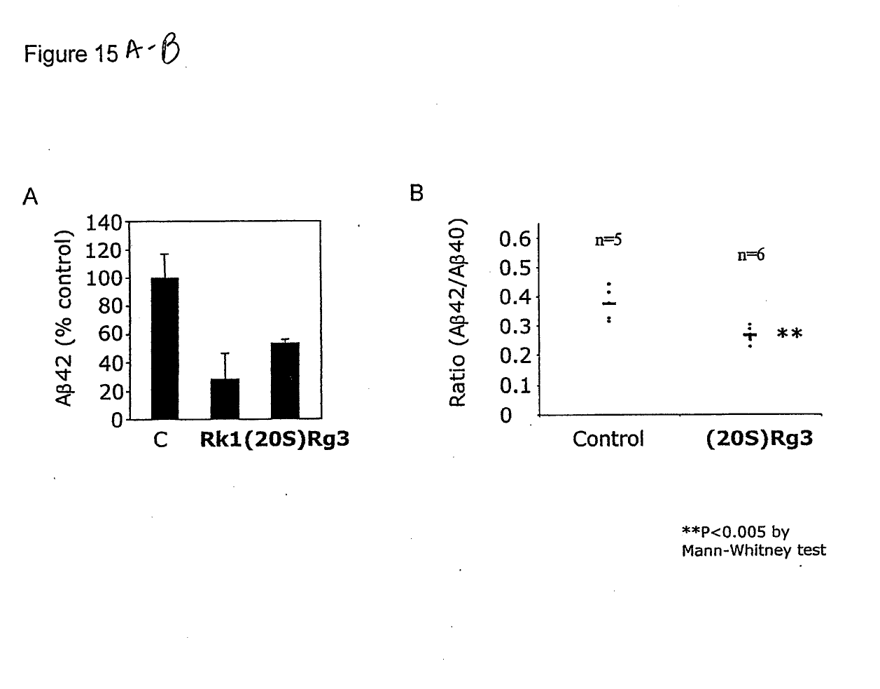

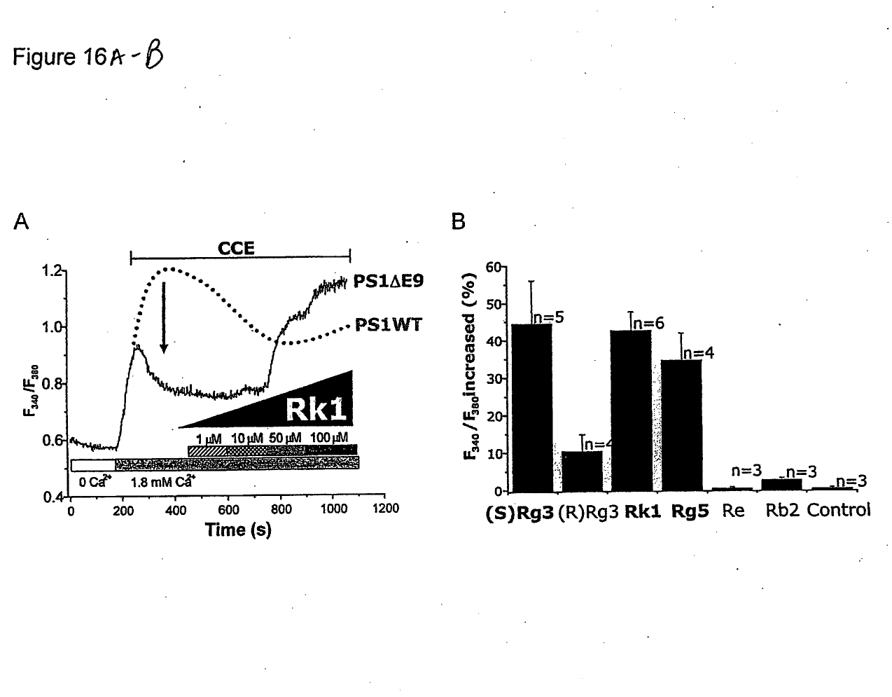

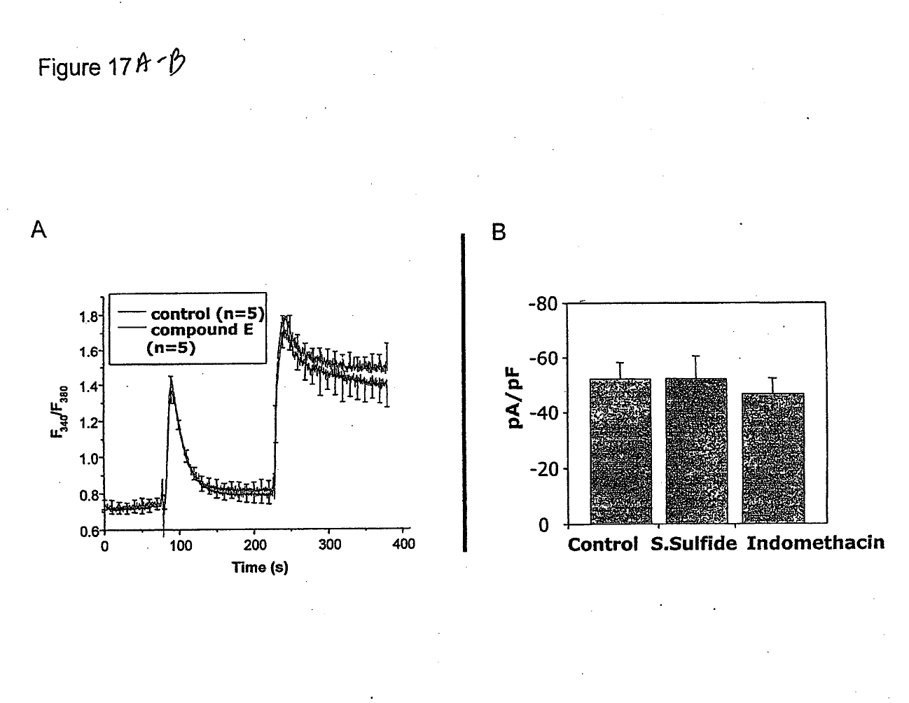

It has been shown that several natural compounds (dammarane triterpenoids) that originate from heat-processed ginseng, including Rk1 and (20S)Rg3, preferentially lower the production of Aβ42 in cell lines and primary neurons (FIG. 14A-C and see United States Patent Application Publication No. 20050245465, Ser. No. 10/834773, by Kim and Chung, published Nov. 3, 2005), with concomitant increase in Aβ37 and Aβ38, by affecting the γ-secretase cleavage step of Aβ42 generation. Administration of an Aβ42-lowering ginsenoside Rg3 results in a decreased Aβ42/Aβ40 ratio in cultured neurons and the brains of a Tg2576 transgenic mouse model of Aβ (FIG. 15A-B). In cell-free assays, these compounds inhibited Aβ generation, while preserving γ-secretase-mediated generation of intracellular domains of APP, Notch and the p75 neurotrophin receptor. Moreover, these Aβ42-lowering natural compounds were able to reverse the cellular cation entry (CCE) deficits associated with presenilin FAD mutant PS1ΔE9 (FIG. 16A-B), suggesting that these compounds directly antagonize the gain-of-function(s) associated with FAD mutant presenilins. Of note, γ-secretase inhibitors and non-steroidal anti-inflammatory drugs were not found to reverse these CCE defects (FIG. 17A-B). Ginsenosides such as (20S)Rg3 may therefore, unlike other Aβ42-lowering agents, also ameliorate the defect in CCE associated with Aβ. The following data support the role of PLCγ1 as a common upstream target modulating CCE as well as Aβ42 levels.

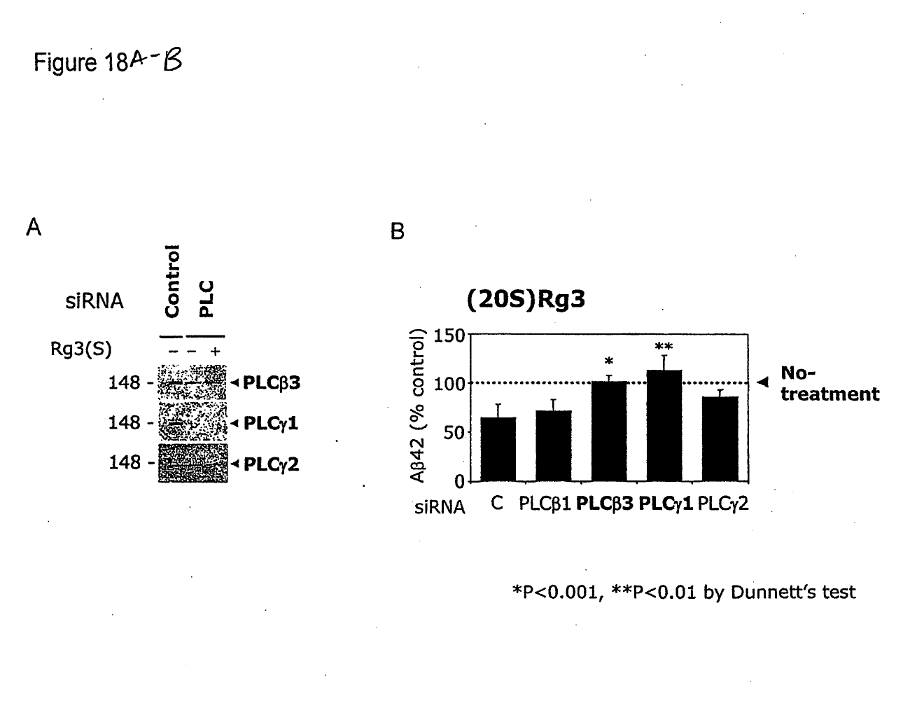

Hela cells stably expressing Swedish FAD mutant APP (Hela-APPsw cells) were treated with small interfering RNA (siRNA) selective against various PLCβ(β1-4) and PLCγ (γ1, 2) isoforms. In Hela-APPsw cells, RT-PCR analysis revealed that PLCβ3, PLCγ1, and PLCγ2 were the major PLC species while other isoforms were detectable but at much lower levels. Treatment of cells with isoform-specific siRNA agents led to an effective suppression of respective PLC isoforms, including PLCβ3, PLCγ1, and PLCγ2, as demonstrated by Western blot analysis (FIG. 18A). When the cells were treated with Rg3, inhibition of PLCβ3 and PLCγ1 expression nearly abolished the Rg3-mediated Aβ42-lowering effect (FIG. 18B). Additional dose-response experiments revealed that, when PLCγ1 levels are suppressed, Rg3 is far less effective in reducing Aβ42 generation, consistent with PLCγ1 being required for the Aβ42-lowering action of ginsenosides.

### 9. REFERENCES

1. Hardy J (1997). Amyloid, the presenilins and Alzheimer's disease. Trends Neurosci. 20, 154-159.
2. Tanzi R E (1999). A genetic dichotomy model for the inheritance of Alzheimer's disease and common age-related disorders. J. Clin. Invest. 104, 1175-1179.
3. Selkoe D J (2001). Alzheimer's disease: genes, proteins, and therapy. Physiol. Rev. 81, 741-766.
4. Selkoe D J and D Schenk (2003). Alzheimer's disease: molecular understanding predicts amyloid-based therapeutics Annu. Rev. Pharmacol. Toxicol. 43, 545-584.
5. Kim S H et al. (2000). Subcellular localization of presenilins: association with a unique membrane pool in cultured cells. Neurobiol. Dis. 7, 99-117.
6. Kaether C et al. (2002). Presenilin-1 affects trafficking and processing of betaAPP and is targeted in a complex with nicastrin to the plasma membrane. J. Biol. 158 (3), 551-561.
7. Thinakaran G et al. (1996). Endoproteolysis or presenilin 1 and accumulation of processed derivatives in vivo. Neuron 17, 181-190.
8. Kim T W et al. (1997). Endoproteolytic processing and proteasomal degradation of presenilin 2 in transfected cells, J. Biol. Chem. 272, 11006-11010.
9. Seeger M et al. (1997). Evidence for phosphorylation and oilgomeric assembly of presenilin 1. Proc. Natl. Acad. Sci. U.S.A. 94, 5090-5094.
10. Kimberly W T et al. (2000). The transmembrane aspartates in presenilin 1 and 2 are obligatory for -secretase activity and amyloid -protein generation. J. Biol. Chem. 275, 3173-3178.
11. Esler W P 35 et al (2000). Transition-state analogue inhibitors of -secretase bind directly to presenilin-1. Nat. Cell. Biol. 2, 1-7.
12. Li Y M et al. (2000). Photoactivated -secretase inhibitors directed to the active site covalently label presenilin 1. Nature 405, 689-693.
13. Wolf M S et al. (1999). Are presenilins intramembrane-cleaving proteases? Implication for the molecular mechanism of Alzheimer's disease. Biochemistry 38, 11223-11230.
14. Scheuner D et al. (1996). Secreted amyloid-protein similar to that in senile plaques of Alzheimer's disease is increased in vivo by the presenilin 1 and 2 and APP mutations linked to familial Alzheimer's disease. Nat. Medicine 2, 864-870.
15. Czech C. Tremp G. and L Pradier (2000). Presenilins and Alzheimer's disease: biological functions and pathogenic mechanisms. Prog Neurobiol. 60(4), 363-384.
16. Etcheberrigaray R. et al. (1998). Calcium responses in fibroblasts from asymptomatic members of Alzheimer's disease families. Neurobiol. Dis, 5, 37-45.
17. Ito E et al. (1994). Internal Ca+2 mobilization is altered in fibroblasts from patients with Alzheimer's disease. Proc. Natl. Acad. Sci. USA 91, 534-538.
18. Leissring M A et al. (2001). Subcellular mechanisms of presenilin-medicated enhancement of calcium signaling. Neurobiol. Dis. 8, 49-478.
19. Guo Q et al. (1996). Alzheimer's PS-1 mutation perturbs calcium homeostatsis and sensitizes PC12 cells to death induced by amyloid b-peptide. Neuroreport 8, 379-383.
20. Schneider et al. (2001). Mutant presenilins disturb neuronal calcium homeostasis in the brain of transgenic mice, decreasing the threshold for excitotoxicity and facilitating long-term potentiation. J. Biol. Chem. 276, 11539-1154.
21. Yoo A S et al. (2000). Presenilin-mediated modulation of capacitative calcium entry. Neuron 27, 561-572.
22. Leissring M A et al. (2000). Capacitative calcium entry deficits and elevated luminal calcium content in mutant presenilin-1 laockin mice. J. Cell Biol. 149, 793-798.
23. Herms J. et al. (2003). Capacitative calcium entry is directly attenuated by mutant presenilin-1, independent of the expression of the amyloid precursor protein. J. Biol. Chem. 278, 2484-2489.
24. Thinakaran G and A T Parent (2004). Identification of the role of presenilins beyond Alzheimer's disease. Pharm. Res. 50, 411-418.
25. Williams R L (1999). Mammalian phosphoinositide-specific phospholipase C. Biochim. Biophys. Acta 1441, 255-267.
26. Rhee S G and Y S Bai (1997). Regulation of phosphoinostitide-specific phospholipase C isozymes. J. Biol. Chem. 272(24), 15045-15048.
27. Katan M (1998). Families of phosphoinositide-specific phospholipase C structure and function. Biochim. Biophys. Acta 1436, 5-17.
28. Kim D et al. (1997). Phospholipase C isozymes selectively couple to specific neurotransmitter receptors. Nature 389, 290-293.
29. Delmas P. Crest M and DA Brown (2004). Functional organization of PLC signaling microdomains in neurons. Trend Neurosci 27(1), 41-47.
30. Hilgemann D W, Feng S and C Nasuhoglu (2001). The complex and intriguing lives of PIP2 with ion channels and transporters, STKE 111, 1-8.
31. Shimohama S et al. (1998). Phospholipase C isozymes in the human brain and their changes in Alzheimer's disease. Neuroscience 82(4), 999-1007.
32. Zhang D et al. (1998). Regional levels of brain phospholipase Cgamma in Alzheimer's disease Brain Res. 811(1-2), 161-165.
33. Ferrari-DiLeo G and D D Flynn (1993). Diminished muscarinic receptor-stimulated [3H]-PIP2 hydrolysis in Alzheimer's disease. Life Sci. 53(25), PL439-444.
34. Crews F T, Kurian P and G Freund (1994). Cholinergic and serotonergic stimulation of phosphoinositide hydrolysis is decreased in Alzheimer's disease. Life Sci. 55(25-26), 1993-2002.
35. De Sarno P et al. (2000). Alterations in muscarinic receptor-coupled phosphoinostitide hydrolysis and Aβ-1 activation in Alzheimer's disease cybrid cells. Neurobiol. Agin 21(1), 31-38.
36. Runnels L W, Yue L, and Clapham D E (2002). The TRPM7 channel is inactivated by PIP2 hydrolysis, Nature Cell Biology 4, 329-336.
37. Wrigley et al. (2005). Functional overexpression of -secretase reveals protease-independent tranfficking functions and a critical role of lipids for protease activity. J Biol. Chem. 280(13), 12523-12535.
38. Wenk M R et al. (2001). PIP kinase 1 is the major PI(4,5)P2 synthesizing enzyme at the synapse. Neuron 32, 79-88.
39. Di Paolo G et al. (2004). Impaired PtdIns(4,5)P2 synthesis in nerve terminals produces defects in synaptic vesicle trafficking. Nature 431, 415-422.
40. Cremona O et al. (1999). Essential role of phosphoinositide metabolism in synaptic vesicle recycling. Cell 99(2), 179-188.
41. Martinat C et al. (2004). Sensitivity to oxidative stress in DJ-1 deficient dopamine neurons: an ES-derived cell model of primary Parkinsonism. PLoS Biology 2 (11), e327.
42. Bibel M et al. (2004). Differentiation of mouse embryonic stem cells into a defined neuronal lineage. Nature Neurosci. 7(9), 1003-1009.
43. Stpyridis M P and A G Smith (2003). Neural differentation of mouse embryonic stem cells. Biochem Soc. Trans. 31(1), 45-49.
44. Xian H Q et al. (2003). Subset of ES-cell-derived neural cells marked by gene targeting. Stem Cells 21, 41-49.
45. Abe Y et al. (2003). Analysis of neurons created from wild-type and Alzheimer's mutation knock-in-embryonic stem cells by a highly efficient differentiation protocol. J. Neurosci 23 (24), 8513-8525.
46. Kim S H et al. (2001). Multiple effects of aspartate mutant presenilin 1 on the processing and trafficking of anyloid precursor protein. J. Biol. Chem. 276(46), 43343-43350.
47. Pendaries C. et al. (2003). Phosphoinositide signaling disorders in human diseases. FEBS Lett. 546(1):25-31.
48. Brewer, G. J. & Cotman, C. W. (1989) Survival and growth of hippocampal neurons in defined medium at low density: advantages of a sandwich culture technique or low oxygen. Brain Res. 494, 65-74.
49. Hurley and Meyer (2001). Subcellular targeting by membrane lipids, Curr. Opin. Cell Biol. 13(2):146-152.
50. Lemon et al. (2003). Metabolic receptor activation, desensitization and sequestration I: modeling calcium and inositol 1,4,5 trisphosphate dynamics following receptor activation. J. Theoret. Biol. 223(1):93-111.
51. Lemon et al. (2003). Metabolic receptor activation, desensitization and sequestration II: modeling calcium and inositol 1,4,5 trisphosphate dynamics following receptor activation. J. Theoret. Biol. 223(1):113-129.
52. McLaughlin et al. (2002). PIP2 and proteins: interactions, organization, and information flow. Annu. Rev. Biophys. Biomol. Struct. 31:151-175.
53. Mili et al. (1996). N_WASP, a novel actin-depolymerizing protein, regulates the cortical cytoskeletal rearrangement in a PIP2 dependent manner downstream of tyrosine kinases. EMBO J. 15(9):5326-5335.
54. Papayannopoulos et al. (2005). A polybasic motif allows N-WASP to act as a sensor of PIP2 density. Mol. Cell. 17(2):181-191.
55. Giantonio B et al. (2004) Phase I and Pharmacokinetic Study of the Cytotoxic Ether Lipid Ilmofosine Administered by Weekly Two-Hour Infusion in Patients with Advanced Solid Tumors. Clinical Cancer Res. 10:1282-1288.
56. Croft S L et al. (1993). Antileishinanial activity of the ether phospholipid ilmofosine. Trans R Soc Trop Med. Hyg. 87(2):217-219.
57. Principe P. et al. (1994). Tumor Cell Kinetics Following Long-Term Treatment with Antineoplastic Ether Phospholipids. Cancer Detection and Prevention 18(5):393-400.
58. Haufe G and Burchardt A (2001). Synthesis of a Fluorinated Ether Lipid Analogous to a Platelet Activating Factor. Eur. J. Organic Cem. 23:4501-4507.
59. Schmid A and Woscholski R (2004). Phosphatases as small molecule target: inhibiting the endogenous inhibitors of kinases. Biochem. Soc. Trans. 32(part 2):348-349.
60. Shingu T et al. (2003). Growth inhibition of human malignant glioma cells induced by the P13-K-specific inhibitor. J. Neurosurg. 98(1): 154-161.
61. Berdel N V et al., (1987). Clinical phase I pilot study of the alkyl lysophospholipid derivative ET-18-OCH₃. Lipids 22(11):967-999.
62. Halet G et al.(2002). The dynamics of plasma membrane PtdIns(4,5)P(2) at fertilization of mouse eggs. J Cell Sci. 115(Pt 10):2139-49.
63. Stokes C E and J N Hawthorne (1987). Reduced phosphoinositide concentration in anterior temporal cortex of Alzheimer-diseased brains. J Neurochem 48(4):1018-21.
64. Lee et al. (1995). Amyloid precursor protein processing is stimulated by metabotropic glutamate receptors. Proc Natl Acad Sci USA 92(17):8083-7.
65. Nitsch R M et al. (1996). Serotonin 5-HT2a and 5-HT2c receptors stimulate amyloid precursor protein ectodomain secretion. J Biol Chem 271 (8):4188-94.
66. Berkovic D (1998). Cytotoxic etherphospholipid analogues. Gen. Pharmacol. 31(4):511-517.
67. Braak H and Braak E (1991). Demonstration of amyloid deposits and neurofibrillary changes in whole brain sections. Brain Pathol. 1(3):213-216.
68. Morrison J H and H of PR (1997). Life and death of neurons in the aging brain. Science 278(5337):412-419.
69. Buxbaum J D, Oishi M, Chen H I, Pinkas-Kramarski R, Jaffe E A, Gandy S E, Greengard P (1992). Cholinergic agonists and interleukin 1 regulate processing and secretion of the Alzheimer beta/A4 amyloid protein precursor. Proc Natl Acad Sci USA, 89(21):10075-8.
70. Nitsch R M, Slack B E, Farber S A, Borghesani P R, Schulz J G, Kim C, Felder C C, Growdon J H, Wurtman R J (1993). Receptor-coupled amyloid precursor protein processing. Ann N Y Acad Sci 695:122-7.
71. Lee R K K, Wurtman R J, Cox A J, Nitsch R M (1995). Amyloid precursor protein processing is stimulated by metabotropic glutamate receptors. Proc Natl Acad Sci USA, 92:8083-7
72. Chung J K, Sekiya F, Kang H S, Lee C, Han J S, Kim S R, Bae Y S, Morris A J, Rhee S G (1997). Synaptojanin inhibition of phospholipase D activity by hydrolysis of phosphatidylinositol 4,5-biphosphate. J Biol Chem 272(25):15980-5
73. Wenk M R, Pellegrini L, Klenchin V A, Di Paolo G, Chang S, Daniell L, Arioka M, Martin T F, De Camilli P (2001) PIP kinase Igamma is the major PI(4,5)P(2) synthesizing enzyme at the synapse. Neuron. 32(1):79-88
74. McLean C A, Chemy R A, Fraser F W, Fuller S J, Smith M J, Beyreuther K, Bush A I, Masters C L (1999). Soluble pool of Aβ amyloid as a determinant of severity of neurodegeneration in Alzheimer's disease, Ann. Neurol. 46: 860-866.
75. Lue L F, Kuo Y M, Roher A B, Brachova L, Shen Y, Sue L, Beach T, Kurth J H, Rydel R E, Rogers J (1999). Soluble amyloid beta peptide concentration as a predictor of synaptic change in Alzheimer's disease, Am. J. Pathol. 155: 853-862.
76. Kayed R, Head E, Thompson J L, McIntire T M, Milton S C, Cotman C W, Glabe C G (2003). Common structure of soluble amyloid oligomers implies common mechanism of pathogenesis, Science 300: 486-489.
77. Wolscholski R, Parker P J (1997). Inositol lipid 5-phosphatases—traffic signals and signal traffic. TIBS 22: 427-431
78. Erneux C, Govaerts C, Communi D, Pesesse X (1998) The diversity and possible functions of the inositol polyphosphate 5-phosphatases. Biochim Biophys Acta 1436:185-199
79. Cremona O et al. (2000). Assignment of SYNJ1 to human chromosome 21q22.2 and Synji2 to the murine homologous region on chromosome 16C3-4 by in situ hybridization. Cytogenet Cell Genet. 88:88-89
80. Arai Y et al. (2002). Excessive expression of synaptojanin in brains with Down's síndrome. Brain and Dev 24:67-72

Various publications are cited herein, the contents of which are hereby incorporated by reference in their entireties.

What is claimed is:

1. A method of reducing Aβ42 generation in a neuronal cell comprising administering, to the neuronal cell, an agent that modulates the activity of an enzyme selected from the group consisting of 5-phosphoinositide phosphatase, phosphoinositide 3 kinase, phosphoinositol phosphate 5-kinase type 1γ, phospholipase C and “phosphatase and tensin homolog deletion on chromosome ten.”

2. The method of claim 1, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

3. The method of claim 1, wherein the agent is selected from the group consisting of edelfosine, miltefosine, perifosine, an erucyl-containing phosphocholine, a brassidyl-containing phosphocholine, an ervonyl-containing phosphocholine, erucylphosphocholine, ilmofosine, BN 52205, BN 5221.1, 2-fluoro-3-hexadecyloxy-2-methylprop-1-yl 2′-(trimethylammonio) ethyl phosphate and LY294002.

4. An assay system for identifying an agent that modulates phosphoinositide levels in a differentiated class of cells, comprising a stem cell that expresses a detectable phosphoinositide sensor, wherein the stem cell is induced to differentiate in order to recapitulate one or more distinguishing feature of the differentiated class of cells.

5. The assay system of claim 4, wherein the phosphoinositide is phosphotidylinositol 4,5-biphosphate.

6. The assay system of claim 4, wherein the differentiated class of cells is pyramidal neurons.

7. The assay system of claim 6, wherein the stem cell is engineered to further contain a gene associated with the development of Alzheimer's disease selected from the group consisting of a mutated presilin 1 gene, a mutated presenilin 2 gene, and a mutated amyloid precursor protein gene.

8. The assay system of claim 7, wherein the distinguishing feature is selected from the group consisting of senile plaques, neurofibrillary tangles, and increased Aβ42.

9. The assay system of claim 8, wherein the phosphoinositide sensor is Pleckstrin homology domain of PLCdelta1 conjugated to green fluorescence protein (PH-GFP).

10. A method of identifying an agent that modulates the level of a phosphoinositide of interest, comprising:
(i) providing a stem cell that expresses a detectable phosphoinositide sensor which binds to the phosphoinositide of interest, wherein the stem cell is induced to differentiate in order to recapitulate one or more distinguishing feature of the differentiated class of cells;
(ii) exposing the differentiated stem cell to a test agent; and
(iii) determining whether exposure to the test agent results in a detectable change in the phosphoinositide sensor;
wherein a change in the phosphoinositide sensor indicates that the test agent modulates the level of the phosphoinositide.

11. The method of claim 10, wherein the phosphoinositide is phosphotidylinositol 4,5-biphosphate.

12. The method of claim 10, wherein the differentiated class of cells is pyramidal neurons.

13. The method of claim 12, wherein the distinguishing feature is selected from the group consisting of senile plaques, neurofibrillary tangles, and increased Aβ42.

14. The method of claim 13, wherein the phosphoinositide sensor is PH-GFP having an affinity for phosphotidylinositol 4,5-biphosphate, such that PH-GFP bound to phosphotidylinositol 4,5-biphosphate is associated with the plasma membrane but unbound PH-GFP localizes in the cytosol.

15. The method of claim 14, wherein the ability of the test agent to increase the amount of PH-GFP in the plasma membrane is tested.

16. The method of claim 15, which is used to identify an agent that may be used to treat Alzheimer's disease, and wherein the ability of the agent to increase the amount of PH-GFP in the plasma membrane indicates that the agent may be used to treat Alzheimer's disease.

17. A method of improving memory comprising administering, to a person in need thereof, an effective amount of an agent that modulates the activity of an enzyme selected from the group consisting of 5-phosphoinositide phosphatase, phosphoinositide 3 kinase, phosphoinositol phosphate kinase type 1γ, phospholipase C and “phosphatase and tensin homolog deletion on chromosome ten.”.

18. The method of claim 17, wherein the person in need thereof is a person diagnosed with a disorder selected from the group consisting of Mild Cognitive Impairment and Alzheimer's disease.

19. The method of claim 17, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

20. The method of claim 18, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

21. The method of claim 17, wherein the agent is selected from group a consisting of edelfosine, miltefosine, perifosine, erucylphosphocholine, hexadecylphosphocholine, ilmofosine, BN 52205, BN 522 1.1, 2-fluoro-3-hexadecyloxy-2-2-methylprop-1-yl 2′-(trimethy lammonio) ethylphosphate, and LY294002.

22. The method of claim 18, wherein the agent is selected from group a consisting of edelfosine, miltefosine, perifosine, erucylphosphocholine, hexadecylphosphocholine, ilmofosine, BN 52205, BN 522 1.1, 2-fluoro-3-hexadecyloxy-2-2-methylprop-1-yl 2′-(trimethy lammonio) ethylphosphate, and LY294002.

23. A method of promoting long term potentiation in a neuronal cell comprising administering, to the neuronal cell, an agent that modulates the activity of an enzyme selected from the group consisting of 5-phosphoinositide phosphatase, phosphoinositide 3 kinase, phosphoinositol phosphate kinase type 1γ, phospholipase C and “phosphatase and tensin homolog deletion on chromosome ten.”.

24. The method of claim 23, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

25. The method of claim 23, wherein the agent is selected from group a consisting of edelfosine, miltefosine, perifosine, erucylphosphocholine, hexadecylphosphocholine, ilmofosine, BN 52205, BN 522 1.1, 2-fluoro-3-hexadecyloxy-2-2-methylprop-1-yl 2′-(trimethy lammonio) ethylphosphate, and LY294002.

26. A method of treating Alzheimer's disease comprising administering, to a person in need thereof, an effective amount of an agent that modulates the activity of an enzyme selected from the group consisting of 5-phosphoinositide phosphatase, phosphoinositide 3 kinase, phosphoinositol phosphate kinase type 1γ, phospholipase C and “phosphatase and tensin homolog deletion on chromosome ten.”.

27. The method of claim 26, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

28. The method of claim 26, wherein the agent is selected from group a consisting of edelfosine, miltefosine, perifosine, erucylphosphocholine, hexadecylphosphocholine, ilmofosine, BN 52205, BN 522 1.1, 2-fluoro-3-hexadecyloxy-2-2-methylprop-1-yl 2′-(trimethy lammonio) ethylphosphate, and LY294002.

29. A method of treating Mild Cognitive Impairment comprising administering, to a person in need thereof, an effective amount of an agent that modulates the activity of an enzyme selected from the group consisting of 5-phosphoinositide phosphatase, phosphoinositide 3 kinase, phosphoinositol phosphate kinase type 1γ, phospholipase C and “phosphatase and tensin homolog deletion on chromosome ten.”.

30. The method of claim 29, wherein the 5-phosphoinositide phosphatase is selected from the group consisting of SynJ1, SynJ2, INPP5P, OCRL, SHIP1, SHIP2, SKIP, PIPP, Pharbin/INPP5E, PTEN, MINPPI, INPPI, SAC1, SAC2, and SAC3.

31. The method of claim 29, wherein the agent is selected from group a consisting of edelfosine, miltefosine, perifosine, erucylphosphocholine, hexadecylphosphocholine, ilmofosine, BN 52205, BN 522 1.1, 2-fluoro-3-hexadecyloxy-2-2-methylprop-1-yl 2′-(trimethy lammonio) ethylphosphate, and LY294002.
